(19) United States
(12) Patent Application Publication
(10) Pub. No.: US 2004/0161747 A1
(43) Pub. Date: Aug. 19, 2004

(54) Method for screening for autoimmune disease by identifying polymorphisms in il-12 p40

(76) Inventor: Grant Morahan, North Fitzroy (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/240,376

(22) PCT Filed: Mar. 27, 2001
(86) PCT No.: PCT/AU01/00340

(30) Foreign Application Priority Data
Mar. 27, 2000 (AU) PQ 6466

(51) Int. Cl. C12Q 1/68

(52) U.S. Cl. 435/6

(57) ABSTRACT

A method of screening mammals for an autoimmune disease or a predisposition to said disease (e.g diabeties). The method consists of identifying polymorphisms in IL-12 p40 and linking them to the disease condition.

### FIELD OF THE INVENTION

The present invention relates generally to a method of screening mammalian animals for a disease condition or a predisposition for the development of a disease condition. More particularly, the present invention provides a method of screening for a disease condition or a predisposition for the development of a disease condition characterised by Th1/Th2 dysregulation. Disease conditions contemplated herein include autoimmune conditions such as, but not limited to, diabetes. The present invention is predicated in part on the determination of the presence of a particular form of IL-12 subunit or linkage between an IL-12 subunit and the disease condition.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that that prior art forms part of the common general knowledge in Australia.

Autoimmune diseases result from the body's immune system mounting an immune response to “self” via the aberrant activation of B cells and/or one or more of the subclasses of T cells. The classes of T cells can be defined broadly according to their requirement for particular molecules encoded by the major histocompatibility complex (MHC), and by their function. The class of T cells restricted by MHC class II molecules are generally referred to as “helper” T (referred to herein as “Th”) cells, and can be further divided into two main subclasses depending on the type of immune response which they mediate. These subclasses are referred to as Th1 and Th2, the former subclass mediating cellular immune response and the later mediating an antibody immune response.

There is increasing evidence that some disease states, including autoimmune diseases, may result from dysregulation of Th1/Th2 status. Insulin dependent diabetes melitis (IDDM), for example, results from the dysregulation of T cells in that they may be mediated by an imbalance towards Th1 and Th2 type responses, respectively. Although a number of immunological influences which affect Th1 and Th2 responses have been identified, such as the influence of the cytokines interleukin-10 and interleukin-12 (herein referred to as “IL-12”), the precise molecular mechanisms of regulating the division of Th cells into these subclasses together with their functional regulation has not been elucidated.

IL-12 is comprised of two subunits—p35 and p40. In work leading up to the present invention, the inventors have identified two allelic variants of the IL-12 p40 subunit. Analysis of distribution of these variants in the population has resulted in the surprising correlation of genetic variation in the IL-12 p40 genes with diseases having a bias in T cell response in terms of the Th subtype of the response. In accordance with the present invention, the inventors have identified a method of screening for an individual with a disease condition or predisposition for the development of a disease condition characterised by Th1/Th2 dysregulation. The developments described herein further facilitate the design of methodology to screen for individuals exhibiting resistance to the development of a disease condition characterised by Th1/Th2 dysregulation. In a further aspect there is now facilitated the development of methods of therapeutically and/or prophylactically treating such disease conditions.

### SUMMARY OF HE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide sequence information prepared using the programme PatentIn Version 2.0, presented herein after the bibliography. Bach nucleotide sequence is identified in the sequence listing by the numeric indicator <210> followed by the sequence identifier (e.g. <201>1, <210>2, etc). The length, type of sequence (DNA, etc) and source organism for each nucleotide sequence is indicated by information provided in the numeric indicator fields <211>, <212> and <213>, respectively. Nucleotide sequences referred to in the specification are defined by the information provided in numeric indicator field <400> followed by the sequence identifier (e.g. <400>1, <400>2, etc).

One aspect of the present invention provides a method of determining the presence of a disease condition or a predisposition for the development of a disease condition in a mammalian animal said method comprising screening for the presence of a form of IL-12 p40 genetic sequence or derivative thereof or its expression product wherein the presence of said form of IL-12 p40 genetic sequence or derivative thereof or its expression product is indicative of the presence of the disease condition or the propensity to develop said disease condition.

Another aspect of the present invention provides a method of determining the presence of a disease condition characterised, exacerbated or otherwise associated with Th1/Th2 dysregulation or a predisposition for the development of a disease condition characterised, exacerbated or other associated with Th1/Th2 dysregulation in a mammalian animal said method comprising screening for the presence of a form of IL-12 p40 genetic sequence or derivatives thereof or its expression product wherein the presence of said form of IL-12 p40 genetic sequence or derivative thereof or its expression product is indicative of the presence of the disease condition or the propensity to develop said disease condition.

Still another aspect of the present invention provides a method of determining the presence of a disease condition characterised, exacerbated or otherwise associated with Th1/Th2 dysregulation or a predisposition for the development of a disease condition characterised, exacerbated or otherwise associated with Th1/Th2 dysregulation in a mammalian animal said method comprising screening for the presence of an allelic form of IL-12 p40 genetic sequence or derivative thereof or its expression product wherein the presence of said allelic form of IL-12 p40 genetic sequence or derivative thereof or its expression product is indicative of the presence of the disease condition or the propensity to develop said disease condition.

Yet another aspect of the present invention provides a method of determining the presence of a disease condition characterised, exacerbated or otherwise associated with Th1/Th2 dysregulation or a predisposition for the development of a disease condition characterised, exacerbated or otherwise associated with Th1/Th2 dysregulation in a mammalian animal said method comprising screening for the presence of the Taq1⁺ allelic form of IL-12 p40 genetic sequence or derivative thereof or its expression product wherein the presence of said Taq1⁺ allelic form of IL-12 p40 genetic sequence or derivative thereof or its expression product is indicative of the presence of the disease condition or the propensity to develop said disease condition.

Even more preferably said IL-12 p40 Taq1⁺ allelic form comprises the nucleotide sequence substantially as set forth in<400>1.

Still yet another aspect of the present invention provides a method of determining the presence of a disease condition characterised, exacerbated or otherwise associated with Th1/Th2 dysregulation or a predisposition for the development of a disease condition characterised, exacerbated or otherwise associated with Th1/Th2 dysregulation in a mammalian animal said method comprising screening for the presence of the Taq1⁻ allelic form of IL-12 p40 genetic sequence or derivative thereof or its expression product wherein the presence of said Taq1 allelic form of IL-12 p40 genetic sequence or derivative thereof or its expression product is indicative of the presence of the disease condition or the propensity to develop said disease condition.

Yet still another aspect of the present invention provides a method of determining the presence of an autoimmune disease condition or a predisposition for the development of an autoimmune disease condition in a mammalian animal said method comprising screening for the presence of an allelic form of IL-12 p40 genetic sequence or derivative thereof or its expression product wherein the presence of said allelic form of IL-12 p40 genetic sequence or a derivative thereof or its expression product is indicative of the presence of said autoimmune disease condition or the propensity to develop said autoimmune disease condition.

A further aspect of the present invention provides a method of determining the presence of IDDM or a predisposition for the development of IDDM in a mammalian animal said method comprising screening for the presence of the Taq1⁻ allelic form of IL-12 p40 genetic sequence or derivative thereof or its expression product wherein the presence of said Taq1⁻ allelic form of IL-12 p40 genetic sequence or derivative thereof or its expression product is indicative of the presence of said IDDM or the propensity to develop said IDDM.

Another further aspect of the present invention provides a method of determining the presence of a disease condition or a predisposition for the development of a disease condition in a mammalian animal said method comprising screening for the presence of a form of IL-12 p40 genetic sequence or derivative thereof or its expression product wherein said IL-12 p40 genetic sequence or derivative thereof is linked to another gene.

Yet another further aspect of the present invention provides a method of determining IDDM or a predisposition for the development of IDDM in a mammalian animal said method comprising screening for the presence of an allelic form of IL-12 p40 genetic sequence or derivative thereof or its expression product wherein said allelic form of IL-12 p40 genetic sequence or derivative thereof is linked to another gene.

Still another further aspect of the present invention provides a method of determining the presence of IDDM or a predisposition for the development of IDDM in a mammalian animal said method comprising screening for the presence of the Taq1⁻ allelic form of IL-12 p40 genetic sequence or derivative thereof or its expression product wherein said Taq1⁻allelic form of IL-12 p40 genetic sequence or derivative thereof is linked to another gene.

Yet still another further aspect of the present invention there is provided a method of determining resistance to a disease condition in a mammal said method comprising screening for the presence of a form of IL-12 p40 genetic sequence or derivative thereof or its expression product wherein the presence of said form of IL-12 p40 genetic sequence or derivative thereof or its expression product is indicative of resistance to developing said disease condition.

Still yet another further aspect of the present invention provides a method of determining resistance to a disease condition characterised, exacerbated or otherwise associated with Th1/Th2 dysregulation in a mammalian animal said method comprising screening for the presence of an allelic form of IL-12 p40 genetic sequence or derivative thereof or its expression product wherein the presence of said allelic form of IL-12 p40 genetic sequence or derivative thereof is indicative of a resistance to developing said disease condition.

Another aspect of the present invention provides a method of determining resistance to IDDM in a mammalian animal said method comprising screening for the presence of the Taq1⁺ allelic form of IL-12 p40 genetic sequence or derivative thereof or its expression product wherein the presence of said Taq1⁺ allelic form of IL-12 p40 genetic sequence or derivative thereof or its expression product is indicative of a resistance to developing IDDM.

Yet another aspect of the present invention there is provided a method of determining resistance to a disease condition in a mammalian animal said method comprising screening for the presence of a form of IL-12 p40 genetic sequence or derivative thereof or its expression product wherein said IL-12 p40 genetic sequence or derivative thereof is linked to another gene.

Still another aspect of the present invention provides a method of determining resistance to IDDM in a mammalian animal said method comprising screening for the presence of the Taq1⁺ allelic form of IL-12 p40 genetic sequence or derivative thereof or its expression product wherein said Taq1⁺ allelic form of IL-12 p40 genetic sequence or derivative thereof is linked to another gene.

The present invention should also be understood to extend to methods of detecting novel IL12 p40 polymorphisms based on the use of familial gene transfer linkage studies.

A kit for determining the presence of a disease condition or a predisposition to the development of a disease condition in a mammalian animal said kit comprising a means of detecting the presence or absence of a form of IL-12p40 genetic sequence or derivative thereof or its expression product.

Another aspect of the present invention provides a kit for determining the presence of a disease condition or a predisposition to the development of a disease condition in a mammalian animal said kit comprising in compartmental form a first compartment adapted to contain an agent for detecting the form of IL-12 p40 genetic sequence or derivative thereof or its expression product and a second compartment adapted to contain reagents useful for facilitating the detection by the agent in the first compartment. Further compartments may also be included, for example, to receive a biological sample. The agent may be an oligonucleotide or antibody or other suitable detecting molecule.

Yet another aspect of the present invention provides a kit for determining resistance to a disease condition in a mammalian animal said kit comprising in compartmental form a first compartment adapted to contain an agent for detecting the form of IL-12 p40 genetic sequence or derivative thereof or its expression product and a second compartment adapted to contain reagents useful for facilitating the detection by the agent in the first compartment. Further compartments may also be included, for example, to receive a biological sample. The agent may be an oligonucleotide or antibody or other suitable detecting molecule.

The present invention further contemplates a method of treatment and/or prophylaxis of the disease conditions herein defined said method comprising administering to a mammal an effective amount of a form of IL-12 p40 genetic sequence or derivative, agonist or antagonist thereof or a molecule which regulates the functioning of said IL-12 p40 genetic sequence or its expression product or derivative, antagonist or agonist thereof wherein said IL-12 p40 or regulatory molecule thereof promotes resistance to said disease condition.

### BRIEF DESCRIPTION OF THE DRAWINGS

FIG. 1 is a schematic representation of a high resolution map of the IL12p40 locus.

A. Placement of IL-12p40 on the radiation hybrid map of chromosome 5q33 relative to the genes GABRA1 (Johnson et al., 1992) and GABRA6 (Hicks et al., 1994) and microsatellite markers (Weissenbach et al., 1992). Oligonucleotides were designed to amplify sequences from the 3′ untranslated region of the human IL-12p40 gene but not from hamster genomic DNA. Radiation hybrids from the Genebridge 4 series (Research Genetics, AL) was tested for human IL-12p40. Additional primers, including GABR41AA (Johnson et al., 1992) and D5S403, D5S410 and D5S412 (Weissenbach et al., 1992) were also tested. The results were used to search against the Whitehead Institute database (http://wwwgenome.wi.mit.edu/cgi-bin/contig/rhmapper.pl) using a LOD=15 for linkage to the framework map.

B. Detailed restriction maps of the PAC and BAC clones containing IL-12p40. A PAC containing IL12p40 was isolated by screening pools from the human PAC library produced by (Ioannou et al., 1994). The direction of transcription of the gene is shown by the arrow. The marker 93/SP6 was obtained from the end sequence of PAC93-1, and used to screen a BAC library. The resulting clone, BAC 626-19, had an 165 kb insert containing the entire PAC93-1 insert (130 kb) with an additional 2.5- and 30-kb at its SP6 and T7 ends, respectively. Restriction enzyme maps were prepared after digestion with NotI, SalI, SacII and MluI, followed by resolution by pulsed field gel electrophoresis, and hybridization with oligonucleotides complementary to the vector ends (T7 or SP6) or to the promoter or 3UTR of IL-12p40.

C. Genomic organisation of the human IL-12p40 gene By comparing the complete genomic sequence with published cDNA sequences, the position of exons 1-8 and introns was deduced. Open boxes=coding exons; first and last exons are non-coding. Size of introns is indicated below the line. Start and stop codons are indicated. The asterisk indicates the presence of a mRNA degradation motif (Zubiaga et al., 1995). Arrows indicate approximate positions of confirmed polymorphisms (see Tables 8, 9 and 10).

FIG. 2 is a schematic representation of the Complete genomic sequence of the IL-12p40 gene (<400>130). The sequence starts 2,397 nucleotides upstream of the TATA box and overlaps the previously published partial promoter sequence. The eight exon sequences (underlined) were determined by comparison with the IL-12p40 cDNA sequences. The translation initiation (ATG) and termination (TAG) codons are double underlined. The 9 base AU-rich element (ARE) consensus sequence is indicated by thick underlining.

FIG. 3 is a graphical representation of linkage of TID to chromosome 5q. Families with at least two affected sibs were genotyped at markers extending over 33 cM of chromosome 5q. Multipoint linkage analysis was undertaken using the MAPMAKER/Sibs software program (Kruglyak et al., 1995). Output shows maximized lod scores (Holmans P., 1993) for all 249 sibpairs from 187 multiplex families (dashed line). MLS scores were also determined for sibpairs who were either identical (HLA IBD) or mismatched (HLA MIS). Dotted line indicates MLS=2.3, which may be taken as significant evidence for linkage in a single test for linkage (Holians P., 1993). Markers were microsatellite repeats (Weissenbach et al., 1992) including the highly polymorphic repeat within the gene GABRA1.

FIG. 4 is a diagramatic representation of TDT of IL12B markers placed on the physical map of 5q33-34.

A. Physical map of YAC (left) and BAC (right) clones containing IL12B. The transcriptional orientation is shown with respect to the centromere. The location of the D5S2937 TAA repeat (box) and the SP6 end (circle) of the PAC clone 93.1 is shown in relation to the genetic markers within the promoter, intron 4 and 3′ UTR of IL12B. Additional anonymous markers on the YAC map are indicated as crosses; the ADRA1b is located telomeric to, and is in the same transcriptional orientation as, IL12B. YAC's shown are (from left): 917b7, 910b3 and 756f1. Further YAC and PAC details were described previously (Huang et al., in press). Restriction sites were determined for Notl (Not), Sacl (Sac), M7ul (Mlu) and Sall (Sal).

B. TDT of IL12B markers. Results from families in which sibs show linkage to IL12B (that is, “IBD 2”) or not (“IBD 1/0”) are shown as the negative log of the P value returned from the TDT. IBD status was assessed by genotypes at the highly polymorphic nearby GABRA4 locus and also at flanking markers. Transmission ratio of the 3′ UTR alleles in IBD 2 families was 76:33 and in IBD 1/0 families was 95:89; of the intron 4 alleles was 61:22 and 76:63; and of the promoter alleles was 44:61 and 132:130, respectively.

FIG. 5 is a diagramatic representation of allele-dependent expression of IL12B.

A. Total RNA was isolated from 1/1 and 2/2 EBV-transformed cell lines and northern analysis was performed with human IL12B and GAPDH cDNA probes.

B. The levels of IL12B MRNA in each cell line relative to GAPDH was determined by densitometry. Bars show mean±s.e. for three separate experiments.

FIG. 6 is a schematic representation of the sequence determination and comparison of IL12p40 promoter alleles in humans.

A. The sequence of the region containing the polymorphism 5′ of the IL-12p40 gene is shown (<400>131). This sequence was determined from a PAC clone we isolated. Underlined=match with published seq (GENBANK HSU89323, Ma et al); bold=oligos used to amplify polymorphism. NB; we have designed another REV oligo to allow for generation of a smaller PCR product. (The first rev oligo is indicated by italics).

B. Alignment of human alleles 1 (<400>132) and 2 (<400>133) showing the complex change involving the insertion of 5 bases and the deletion of a G resulting in a nett gain of 4 bases compared to the shorter allele 2. No other differences were found in a total of 2 kb sequenced upstream of the IL-12p40 gene.

### DETAILED DESCRIPTION OF THE PREFERRED EMBODIMENTS

The present invention is predicated, in part, on the identification of allelic variants of an IL 12 subunit and more particularly p40 subunit of IL-12 and the surprising observation that a correlation exists between the expression of a particular genetic variant and the onset of an autoimmune disease condition such as, but not limited to, IDDM. Although not intending to limit the invention to any one theory or mode of action, it is proposed that genetic variation in the IL-12 p40 gene modulates the expression levels of the RNA thereby modulating the levels of the IL-12 protein and thereby biasing the Th cell response either towards a Th2 type response or a Th1 type response. This proposed mechanism of action now provides a means for the development of a method of screening individuals to determine a predisposition to developing diseases involving the dysregulation of the Th1/Th2 response and or resistance thereto a means for the rational design of therapeutic or prophylactic regimes and/or molecules for modulation of the Th1/Th2 response.

Accordingly, one aspect of the present invention provides a method of determining the presence of a disease condition or a predisposition for the development of a disease condition in a mammalian animal said method comprising screening for the presence of a form of IL-12 p40 genetic sequence or derivative thereof or its expression product wherein the presence of said form of IL-12 p40 genetic sequence or derivative thereof or its expression product is indicative of the presence of the disease condition or the propensity to develop said disease condition.

More particularly the present invention provides a method of determining the presence of a disease condition characterised, exacerbated or otherwise associated with Th1/Th2 dysregulation or a predisposition for the development of a disease condition characterised, exacerbated or other associated with Th1/Th2 dysregulation in a mammalian animal said method comprising screening for the presence of a form of IL-12 p40 genetic sequence or derivatives thereof or its expression product wherein the presence of said form of IL-12 p40 genetic sequence or derivative thereof or its expression product is indicative of the presence of the disease condition or the propensity to develop said disease condition.

The term “mammalian animal” includes humans, primates, livestock animals (e.g. horses, cattle, sheep, pigs, donkeys) laboratory test animals (e.g. mice, rats, rabbits, guinea pigs) companion animals (e.g. dogs, cats) and captive wild animals (e.g. kangaroos, deer, foxes). Preferably, the mammal is a human or a laboratory test animal. Even more preferably the mammal is a human.

IL-12 is a heterodimeric glycoprotein composed of unrelated subunits of 35 kDa (p35) and 40 kDa (p40). Accordingly, reference to “IL-12 p40 genetic sequence” should be understood as a reference to all forms of DNA- and RNA encoding (i) all or part of the p40 subunit of IL-12 and derivatives thereof or (ii) all or part of a regulatory sequence (such as a promoter sequence) which directly or indirectly regulates the expression of the IL-12 p40 subunit and is located at a position other than between the IL-12 p40 genomic DNA transcription initiation and termination sites and derivatives thereof.

This definition includes, but is not limited to, all forms of the IL-12 p40 genomic DNA sequence, for example:

(i) allelic variants such as the Taq1⁺ (<400>1) and Taq1⁻ (<400>2), allelic forms which are defined on the basis of the presence of a deoxycytosine or deoxyadenine nucleotide, respectively, at position 235 of <400>1 and <400>2. <400>1 and <400>2 are partial IL-12 p40 cDNA sequences and depict the 3′ end of the IL-12 p40 cDNA. Position 235 occurs in the 3′ untranslated region of the cDNA sequence.

(ii) allelic variants such as those characterised by promotor region polymorphisms (<400>3, <400>4, <400>5, <400>6, <400>7, <400>8, <400>132, <400>133).

(iii) allelic variants such as those characterised by polymorphisms in exon 6 (<400>9, <400>10), exon 7 (<400>11, <400>12) or exon 8 (<400>13, <400>14).

(iv) allelic variants such as those characterised by polymorphisms in intron 1 (<400>41<400>48), intron 2 (<400>49-<400>52), intron 4 (<400>55, <400>58) and intron 7 (<400>59, <400>60).

(v) allelic variants characterised by the presence of any one or more of the polymorphisms detailed in (i)-(iv) all forms of the RNA transcribed from said IL-12 p40 genomic DNA sequence (for example the primary RNA transcript, mRNA or splice variants of the RNA transcript) and the cDNA generated from RNA transcribed from said IL-12 p40 genetic sequence.

Preferably said IL-12 p⁴0 is the Taq1⁺ and/or Taq1⁻ allelic form.

According to this preferred embodiment the present invention provides a method of determining the presence of a disease condition characterised, exacerbated or otherwise associated with Th1/Th2 dysregulation or a predisposition for the development of a disease condition characterised, exacerbated or otherwise associated with Th1/Th2 dysregulation in a mammalian animal said method comprising screening for the presence of an allelic form of IL-12 p40 genetic sequence or derivative thereof or its expression product wherein the presence of said allelic form of IL-12 p40 genetic sequence or derivative thereof or its expression product is indicative of a propensity to develop said disease condition.

More preferably the present invention provides a method of determining the presence of a disease condition characterised, exacerbated or otherwise associated with Th1/Th2 dysregulation or a predisposition for the development of a disease condition characterised, exacerbated or otherwise associated with Th1/Th2 dysregulation in a mammalian animal said method comprising screening for the presence of the Taq1⁺ allelic form of IL-12 p40 genetic sequence or derivative thereof or its expression product wherein the presence of said Taq1⁺ allelic form of IL-12 p40 genetic sequence or derivative thereof or its expression product is indicative of a propensity to develop said disease condition.

Even more preferably said IL-12 p40 Taq1⁺ allelic form comprises the nucleotide sequence substantially as set forth in<400>1.

In another preferred embodiment the present invention provides a method of determining the presence of a disease condition characterised, exacerbated or otherwise associated with Th1/Th2 dysregulation or a predisposition for the development of a disease condition characterised, exacerbated or otherwise associated with Th1/Th2 dysregulation in a mammalian animal said method comprising screening for the presence of the Taq1⁻ allelic form of IL-12 p40 genetic sequence or derivative thereof or its expression product wherein the presence of said Taq1⁻ allelic form of IL-12 p40 genetic sequence or derivative thereof or its expression product is indicative of a propensity to develop said disease condition.

Even more preferably said IL-12 p40 Taq1⁻ allelic form comprises the nucleic acid sequence substantially as set forth in<400>2.

It should be understood that the presence of the Taq1⁺ or Taq1⁻ polymorphism may be indicative of a number of disease conditions characterised by Th1/Th2 dysregulation. In one embodiment, to the extent that the disease condition is IDDM, Taq1⁻ expression in an individual is indicative of a propensity to develop IDDM while Taq+expression in an individual is indicative of resistance to the development of IDDM.

Reference to “expression product” should be understood as a reference to the peptide, polypeptide or protein resulting from the translation of IL-12 p40 RNA sequences or transcription and translation of IL-12 p40 DNA sequences as hereinbefore defined.

Reference to a disease condition “characterised, exacerbated or otherwise associated with Th1/Th2 dysregulation” should be understood as a reference to a disease condition in which at least some of the pathology associated with said disease condition is either directly or indirectly due to the activation of a particular subpopulation of Th cells. For example, IDDM is characterised by a Th1 type response. Preferably, said disease condition is an autoimmune disease condition.

Accordingly, another aspect of the present invention provides a method of determining the presence of an autoimmune disease condition or a predisposition for the development of an autoimmune disease condition in a mammalian animal said method comprising screening for the presence of an allelic form of IL-12 p40 genetic sequence or derivative thereof or its expression product wherein the presence of said allelic form of IL-12 p40 genetic sequence or a derivative thereof or its expression product is indicative of the presence of said autoimmune disease or the propensity to develop said autoimmune disease condition.

Preferably, said autoimmune disease condition is an autoimmune disease condition characterised, exacerbated or otherwise associated with Th1/Th2 dysregulation.

Even more preferably, said allelic form of IL-12 p40 is the Taq1⁺ or Taq1⁻ form.

Without limiting the present invention to any one theory or mode of action, disease conditions characterised by Th1/Th2 dysregulation are thought to be mediated by an imbalance in the Th response in that it is incorrectly skewed towards either a Th1 or Th2 response. The skewing of Th cells towards either a Th1 or a Th2 response is now envisaged as at least partly regulated by genetic variation in the IL-12 p40 gene which acts to modulate the expression levels of the IL-12 p40 polypeptide. Analysis of the frequency of Taq1⁺ and Taq1⁻ IL-12 p40 alleles in subjects who exhibit symptoms of IDDM indicates that expression of a Taq1⁻ allele is indicative of susceptibility to IDDM while expression of a Taq1⁺ allele is indicative of resistance to IDDM.

According to this most preferred embodiment, the present invention provides a method of determining the presence of IDDM or a predisposition for the development of IDDM in a mammalian animal said method comprising screening for the presence of the Taq1⁻ allelic form of IL-12 p40 genetic sequence or derivative thereof or its expression product wherein the presence of said Taq1⁻ allelic form of 1L-12 p40 genetic sequence or derivative thereof or its expression product is indicative of the presence of said IDDM or the propensity to develop said IDDM.

The present invention should be understood to extend to methods of determining the presence of a disease condition characterised, exacerbated or otherwise associated with Th1/Th2 dysregulation, or a predisposition thereof, by screening for the combination of a Taq1⁺/Taq1⁻ polymorphism together with any other polymorphism expressed by an individual.

Still without limiting the invention to any one theory or mode of action, transmission disequilibrium studies have further indicated that in certain disease conditions characterised by Th1/Th2 dysregulation the Taq1⁺ and Taq1⁻ allelic forms of IL-12 p40 are indicative of a predisposition to developing said disease when they have been transmitted to the affected mammal in a form where they are linked to another gene. The method of the present invention is exemplified herein utilising the genetic marker GABRAL which occurs in two allelic forms—A and B. Expression of the Taq1⁻/GABRA1-A haplotype where the two genes are linked is indicative of IDDM susceptibility while expression of the Taq1⁻/GABRA1-A haplotype where the two genes are unlinked is not indicative of IDDM susceptibility. Conversely, transmission of the Taq1⁺/GABRA-A haplotype in a linked form is indicative of IDDM resistance.

Accordingly, a related aspect of the present invention provides a method of determining the presence of a disease condition or a predisposition for the development of a disease condition in a mammalian animal said method comprising screening for the presence of a form of IL-12 p40 genetic sequence or derivative thereof or its expression product wherein said IL-12 p40 genetic sequence or derivative thereof is linked to another gene.

Reference to genes being “linked” is a reference to any two or more genes which do not assort independently at meiosis. Determining the linkage of two genes can be achieved by any one of a number of methods including for example screening one or more parents of said mammal to determine the pattern of gene transmission and thereby the degree of linkage between the IL-12 p40 gene or derivative thereof and another gene. Alternatively, said linkage can be determined by screening one or more parents and comparing with a proband.

Preferably said form of IL-12 p40 is an allelic form of IL-12 p40.

In a most preferred embodiment, said disease condition is an autoimmune disease condition and most preferably IDDM.

According to this most preferred embodiment the present invention provides a method of determining IDDM or a predisposition for the development of IDDM in a mammalian animal said method comprising screening for the presence of an allelic form of IL-12 p40 genetic sequence or derivative thereof or its expression product wherein said allelic form of IL-12 p40 genetic sequence or derivative thereof is linked to another gene.

Most preferably the present invention provides a method of determining the presence of IDDM or a predisposition for the development of IDDM in a mammalian animal said method comprising screening for the presence of the Taq1⁻ allelic form of IL-12 p40 genetic sequence or derivative thereof or its expression product wherein said Taq1⁻ allelic form of IL-12 p40 genetic sequence or derivative thereof is linked to another gene.

Preferably, the gene to which said IL-12 p40 genetic sequence is linked is an informative genetic marker. By “informative” it is meant a genetic marker which when used in conjunction with said IL-12 p40 genetic sequence improves or otherwise indicates involvement of said IL-12 p40 genetic sequence in the subject disease or other condition. Preferably, said informative genetic marker is a GABRA allele.

Even more preferably, said other gene is the GABRA1-A allele genetic marker.

The expression of a particular form of IL-12 p40 is also indicative of a mammal's resistance to developing a disease condition characterised by Th1/Th2 dysregulation.

Accordingly, in another aspect of the present invention there is provided a method of determining resistance to a disease condition in a mammal said method comprising screening for the presence of a form of IL-12 p40 genetic sequence or derivative thereof or its expression product wherein the presence of said form of IL-12 p40 genetic sequence or derivative thereof or its expression product is indicative of resistance to developing said disease condition.

Preferably, said disease condition is characterised, exacerbated or otherwise associated with Th1/T2 dysregulation.

More preferably said form of IL-12.p40 genetic sequence is an allelic form of IL-12 p40 genetic sequence.

According to this most preferred embodiment the present invention provides a method of determining resistance to a disease condition characterised, exacerbated or otherwise associated with Th1/Th2 dysregulation in a mammalian animal said method comprising screening for the presence of an allelic form of IL-12 p40 genetic sequence or derivative thereof or its expression product wherein the presence of said allelic form of IL-12 p40 genetic sequence or derivative thereof is indicative of a resistance to developing said disease condition.

Most preferably said allelic form of IL-12 p40 is the Taq1⁺ or Taq1⁻ allelic form.

In a most preferred embodiment said disease condition is an autoimmune disease condition and even more preferably IDDM.

According to this most preferred embodiment the present invention provides a method of determining resistance to IDDM in a mammalian animal said method comprising screening for the presence of the Taq1⁺ allelic form of IL-12 p40 genetic sequence or derivative thereof or its expression product wherein the presence of said Taq1⁺ allelic form of IL-12 p40 genetic sequence or derivative thereof or its expression product is indicative of a resistance to developing IDDM.

In another related aspect of the present invention there is provided a method of determining resistance to a disease condition in a mammalian animal said method comprising screening for the presence of a form of IL-12 p40 genetic sequence or derivative thereof or its expression product wherein said IL-12 p40 genetic sequence or derivative thereof is linked to another gene.

Preferably, said disease condition is characterised, exacerbated or otherwise associated with Th1[Fh2 dysregulation. Even more preferably said disease condition is an autoimmune disease condition and most preferably IDDM.

Still more preferably said IL-12 p40 is the Taq1⁺ allelic form.

According to this most preferred embodiment the present invention provides a method of determining resistance to IDDM in a mammalian animal said method comprising screening for the presence of the Taq1⁺ allelic form of IL-12 p40 genetic sequence or derivative thereof or its expression product wherein said Taq1⁺ allelic form of IL-12 p40 genetic sequence or derivative thereof is linked to another gene.

Most preferably said other gene is GABRA1-A allele genetic marker.

Reference to detecting “resistance” should be understood to generally refer to detecting a reduction in the pathology associated with an existing disease condition, preventing, delaying or minimising the onset of pathology associated with the onset of said disease condition, or preventing the onset of said disease condition.

The present invention should also be understood to extend to methods of detecting novel IL-12 p40 polymorphisms based on the use of familial gene transfer linkage studies. Further sequence polymorphisms may exist in the vicinity of the IL-12 p40 gene. Some of these may also be involved in regulating IL12 p40 gene expression and therefore the ability to produce Th1 or Th2 dominated immune response and hence resistance or susceptibility to autoimmune disease. Such polymorphisms may be tested by their co-segregation with the IL-12 p40 Taq− allele to IDDM subjects, or by their non-transmission in linkage with the IL12 p40 Taq+ allele. An example of how such an additional polymorphism may be detected and its utility is provided with reference to the GABRA-A genotype in Table 6. Such additional polymorphisms may be tested in, for example, functional assays by in vitro transfection experiments using appropriate reporter constructs.

Screening of the forms of IL-12 p40 genetic sequences or derivatives thereof or its expression products may be achieved utilizing any of a number of techniques including PCR analysis and antibody binding assays.

In one preferred method, the IL-12 p40 gene or transcribed RNA is subjected to PCR or RT-PCR, respectively, using primers homologous to gene sequences located 5′ and 3′ of the Taq1 polymorphism. The oligonucleotide is generally labelled with a reporter molecule capable of giving an identifiable signal such as a radioisotope, chemiluminesce molecule or a fluorescent molecule. A particularly useful reporter molecule is a biotinylated molecule.

Another useful detection system involves antibodies directed to the various forms of IL-12 p40 genetic sequences, to the Taq1 polymorphism itself or to the expression products of the various IL-12 p40 forms. Detection utilising antibodies may be accomplished immunologically in a number of ways such as by Western Blotting and ELISA procedures. These procedures include both single site and two site or “sandwich” assays of the noncompetitive type, as well as the traditional competitive binding assays. These assays also include direct binding of a labelled antibody to a target.

Another aspect of the present invention provides a kit for determining the presence of a disease condition or a predisposition to the development of a disease condition in a mammalian animal said kit comprising a means of detecting the presence or absence of a form of IL-12 p40 genetic sequence or derivative thereof or its expression product.

Without limiting this aspect of the present invention in any way, the subject kit may be designed to detect either the presence of a given allele, or its absence, in an individual. In a preferred embodiment the presence of a specific allele is screened for. The means by which the subject kit detects the form of IL-12 p40 may be any suitable means including, but not limited to, any mass spectrometry technique, gels, DNA or protein chips, DNA probing means, antibody or other immunological reagent.

In one embodiment the present invention provides a kit for determining the presence of a disease condition or a predisposition to the development of a disease condition in a mammalian animal said kit comprising in compartmental form a first compartment adapted to contain an agent for detecting the form of IL-12 p40 genetic sequence or derivative thereof or its expression product and a second compartment adapted to contain reagents useful for facilitating the detection by the agent in the first compartment. Further compartments may also be included, for example, to receive a biological sample. The agent may be an oligonucleotide or antibody or other suitable detecting molecule.

In another embodiment the present invention provides a kit for determining resistance to a disease condition in a mammalian animal said kit comprising in compartmental form a first compartment adapted to contain an agent for detecting the form of IL-12 p40 genetic sequence or derivative thereof or its expression product and a second compartment adapted to contain reagents useful for facilitating the detection by the agent in the first compartment. Further compartments may also be included, for example, to receive a biological sample. The agent may be an oligonucleotide or antibody or other suitable detecting molecule.

The present invention further contemplates a method of treatment and/or prophylaxis of the disease conditions hereinbefore defined said method comprising administering to a mammal an effective amount of a form of IL-12 p40 genetic sequence or derivative, agonist or antagonist thereof or its expression product or derivative, antagonist or agonist thereof wherein said IL-12 p40 promotes resistance to said disease condition. For example, in patients suffering from IDDM or a predisposition to developing IDDM the Taq1⁺ form of the IL-12 p40 gene or transcription or translation product or molecules which regulate Taq1⁺ functioning or expression may be administered. The present invention facilitates modulation of the immune system response both in disease states or in non-disease states where it is nevertheless desirable (for example, to regulate IL-12 levels as part of a vaccination protocol).

Administration of said IL-12 p40 can be achieved via one of several techniques including, but in no way limited to:

(i) Introduction of a nucleic acid molecule encoding a particular form of IL-12 p40 or a derivative thereof to modulate the capacity of that cell to synthesize said IL-12 p40.

(ii) Introduction into a cell of a proteinaceous IL-12 p40 molecule of particular form or derivative thereof.

The present invention may be used for the screening of individuals, families and populations. In this regard, the inventors have determined that the relationship between Taq1 allele expression and IDDM resistance or susceptibility is particularly evident in individuals who are ethnically of Northern European or United Kingdom origin. Accordingly, in a preferred embodiment the methods of the present invention are directed to screening individuals of this ethnic origin.

Further features of the present invention are more fully described in the following nonlimiting Examples. It is to be understood, however, that this detailed description is included solely for the purposes of exemplifying the present invention. It should not be understood in any way as a restriction on the broad description of the invention as set out above.

### EXAMPLE 1

### Detection of IL-12 Taq Polymorphism

A polymorphism was found in the 3′ UT region of the IL-12 p40 gene. This polymorphism was detected as follows. DNA was obtained from peripheral blood lymphocytes using standard techniques, and used to initiate polymerase chain reaction (PCR) using synthetic oligonucleotides and Taq DNA polymerase (Gibco). The sequences of these oligos were as follows:

| | | |
|---|---|---|
| FORWARD | TAGCTCATCTTGGAGCGAAT | (<400>134) |
| REVERSE | AACATTCCATACATCCTGGC | (<400>135) |

Reverse oligo hybridises to the following sequence in 3′ UT region:

| | |
|---|---|
| GCCAGGATGTATGGAATGTT | (<400>136) |

Following PCR, aliquots of the reaction products were incubated with Taq1 restriction enzyme (Promega) under conditions suggested by the manufacturer. The samples were then ran on gels (either agarose gels, or acrylamide gels if the primer was first labelled with 32p ATP) to determine the lengths of the DNA fragments. Using the above primers, a product of approximately 0.3 kbp is generated; if the Taq1 site is present, this yields fragments of approximately 0.14 and 0.16 kbp after digestion. Allele 1 is designated as the allele not digested by Taq1; allele 2 contains the Taq1 site (i.e. TCGA).

Other methods for detecting this polymorphism include use of different oligonucleotides flanking the Taq1 site; use of allele-specific primers to preferentially amplify allele 1 (Taq1⁻ polymorphism) or allele 2 (Taq1⁺ polymorphism) sequences; testing products by hybridisation using allele-specific oligonucleotides; testing products or fragments derived therefrom for differences in mass by appropriate methods, e.g. mass spectrometry.

### EXAMPLE 2

### Determining the Frequency of Alleles 1 and 2 in Control Subjects

The frequency of alleles 1 and 2 in controls was determined by typing DNA samples from anonymous donors.

### EXAMPLE 3

IL-12 Allele Expression and IDDM Susceptibility

The role of IL-12 p40 alleles in insulin-dependent diabetes mellitus (IDDM) was tested by determining whether either allele was preferentially transmitted to affected offspring.

These alleles were typed as described in Example 2. Transmission or nontransmission of these alleles from appropriate parents to affected offspring was determined using the Transmission Disequilibrium test (TDT) in the Genetic Analysis System programs (A. Young, GAS Manual User Guide v1.2 (Oxford University, 1995). Results for families are shown in table 1.

TABLE 1
Transmission of IL-12 p40 alleles in IDDM families

| Allele | | | |
|---|---|---|---|
| Both parents | Trans | Not | Prob (binom) |
| 1 | 159 | 110 | 0.0017 |
| 2 | 110 | 159 | |

Transmission Dis-equilibrium Test for Affected Children (not weighted)

These results show that allele 1 is preferentially transmitted and allele 2 is preferentially not transmitted to IDDM offspring.

### EXAMPLE 4

Confirmation of the use of the Taq allele as an indicator of susceptibility to IDDM was obtained from an independent sample of 238 families recruited through the Royal Melbourne and Royal Children's Hospitals.

TABLE 2

| Allele | Trans | Not Trans | p |
|---|---|---|---|
| 1 | 58 | 32 | 0.004 |
| 2 | 32 | 58 | 1 |

These data indicate that allele 1 is preferentially transmitted (i.e. confers susceptibility or is in linkage disequilibrium with the polymorphism that confers susceptibility) and that allele 2 is preferentially not transmitted (i.e. confers resistance).

When these data are analysed with respect to ethnic origin, the following is found:

TABLE 3
North European ethnicity

| Allele | Trans | Not Trans | p |
|---|---|---|---|
| 1 | 49 | 18 | 9.7e−05 +++ |
| 2 | 18 | 49 | 1 |

TABLE 4
Non-North European ethnicity

| Allele | Trans | Not Trans | p |
|---|---|---|---|
| 1 | 9 | 14 | 0.8 |
| 2 | 14 | 9 | 0.2 |

This suggests that this gene is particularly indicative of fDDM in individuals who ethnically originate from the UK and northern Europe.

Pooling the Example 3 data together with the Example 4 data indicates that the total p value in all families (i.e. unselected for linkage to GABRA) is 6×10⁻⁶.

### EXAMPLE 5

### Linked IL-12 Allele Transmission and IDDM Susceptibility

A linked genetic marker in the GABRA-A receptor α1-subunit gene (GABRA1) was also typed. The GABRAL alleles were detected as described by Johnson, K J, et al Genomics 14:745-8. These alleles were subsequently simplified for the transmission disequilibrium analysis as they were found to fall into two distinct groups: the six highest MW alleles were designated as “A” and the three lowest were designated as “B”.

DNA was analysed from families in which at least two children had IDDM. Linkage was assessed by evaluating whether the affected sibs had inherited GABRA1 and linked genes identical-by-descent (IBD) i.e. whether they had inherited the same maternal and the same paternal alleles at GABRA1 and/or at other flanking markers. IBD status for a particular chromosomal region suggests that affected sibs share gene(s) which influence disease susceptibility; such sibs are said to show genetic linkage to the markers shared IBD). Two groups could thus be defined: those who were IBD at the IL-12 p40/GABRA region, and those who were not IBD. Transmission of alleles 1 and 2 were evaluated in sibs showing linkage to GABRA1/IL-12 p40. Families showing no linkage to IL-12 p40 did not show preferential transmission of either allele. Families whose affected sibs showed linkage to IL-12 p40/GABRA1 showed preferential transmission of allele 1, and preferential non-transmission of the other allele. This indicated that these alleles were associated with IDDM susceptibility and resistance, respectively (Table 5).

TABLE 5
Transmission of IL-12 p40 alleles in sibs
showing linkage to IL-12 p40.

| Allele | | | |
|---|---|---|---|
| Both Parents | Trans | Not | Prob (binom) |
| 1 | 102 | 61 | 0.00082 |
| 2 | 61 | 102 | |

Transmission Dis-equilibrium Test for Affected Children (not weighted)

By considering the IL-12 alleles and the linked GABRA1 marker, four haplotypes were defined, as follows. Haplotype 1A, IL-12 p40 allele 1, GABRA1 A; haplotype 1B, IL-12 p40 allele 1, GABRAL B; haplotype 2A, IL-12 p40 allele 2, GABRA1 A; haplotype 2B, IL-12 p40 allele 2, GABRA1 B. Transmission or nontransmission of these haplotypes from appropriate parents to affected offspring was determined using the Transmission Disequilibrium test (IDT) in the Genetic Analysis System programs (A. Young, GAS Manual User Guide v1.2 (Oxford University, 1995).

Families showing no linkage to IL-12 p40 did not show preferential transmission of any haplotype. Families whose affected sibs showed linkage to IL-12 p40/GABRA1 (suggesting that IL-12 p40 may be contributing to their development of IDDM) showed preferential transmission of one haplotype, and preferential non-transmission of another haplotype. This indicated that these haplotypes were associated with IDDM susceptibility and resistance, respectively. (Table 6). Two other haplotypes showed no deviation in transmission, suggesting that they were neutral in conferring susceptibility.

TABLE 6
Transmission Disequilibrium Test of IL-12 haplotypes in IDDM.

| Linkage | | | | |
|---|---|---|---|---|
| Status | Haplotype | Trans | Not Trans | P |
| Unlinked | 1A | 84 | 80 | — |
| | 1B | 74 | 67 | — |
| | 2A | 22 | 27 | — |
| | 2B | 25 | 29 | — |
| Linked | 1A | 94 | 52 | 0.00032 |
| | 1B | 62 | 66 | — |
| | 2A | 8 | 34 | 0.000006 |
| | 2B | 24 | 36 | — |

p determined by Chi² test for deviation from expected 50% transmitted to 50% non-transmitted.

### EXAMPLE 6

### Complete Primary Structure, Chromosomal Localisation and Definition of Polymorphisms of the Gene Encoding the Human IL-12p40 Subunit

High Resolution Mapping

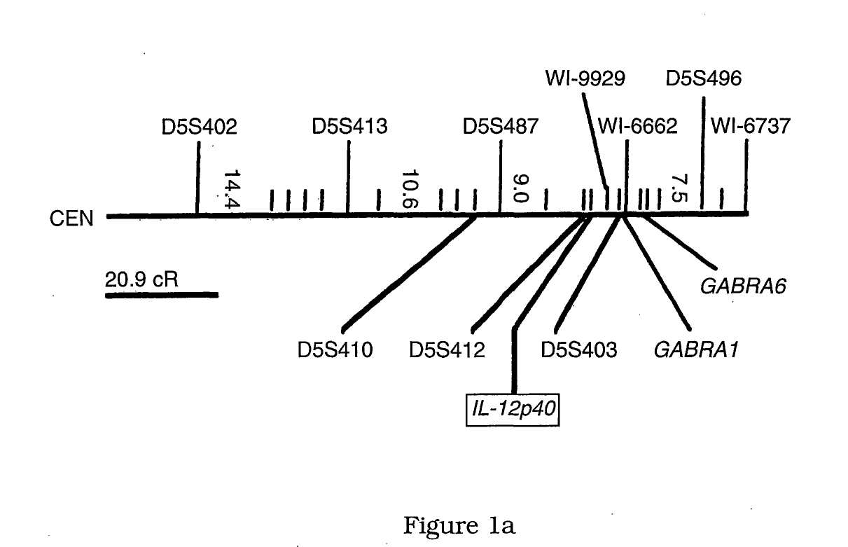

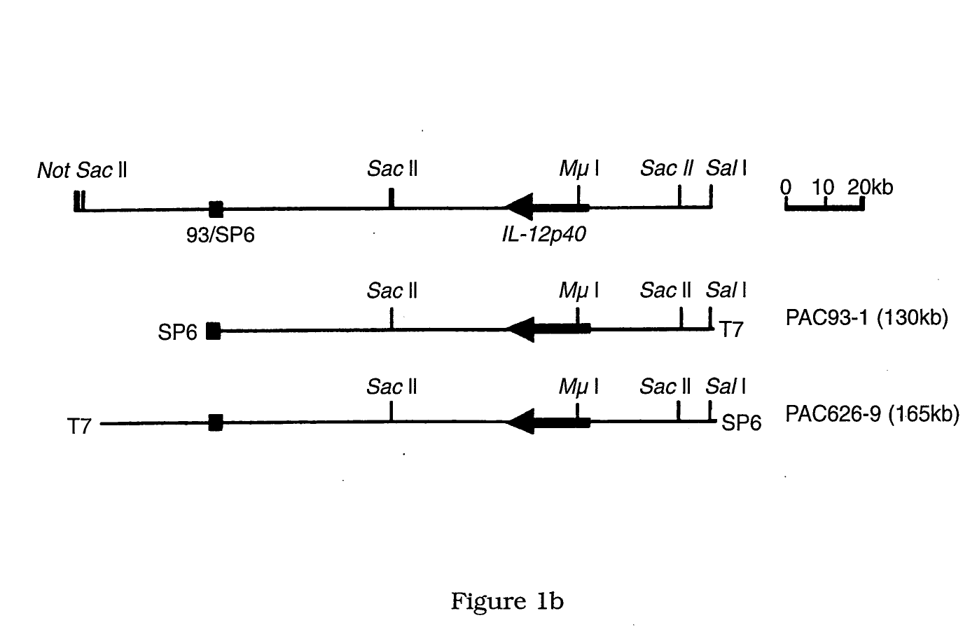

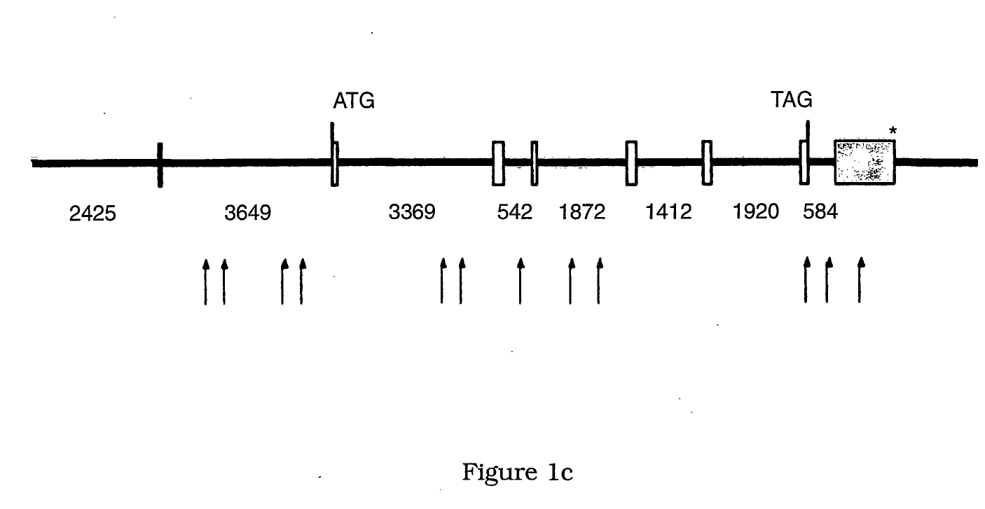

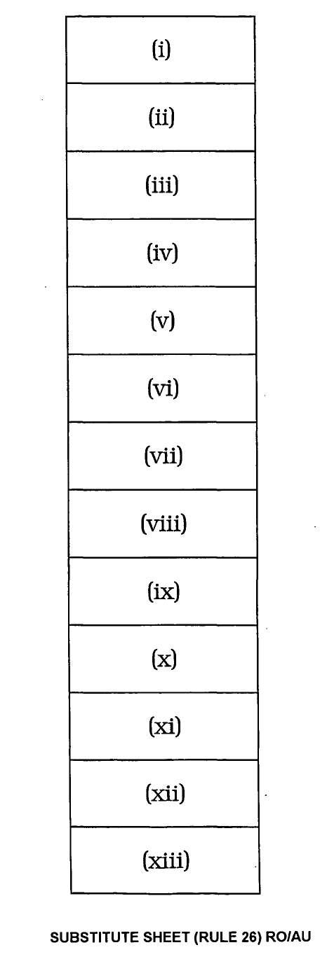

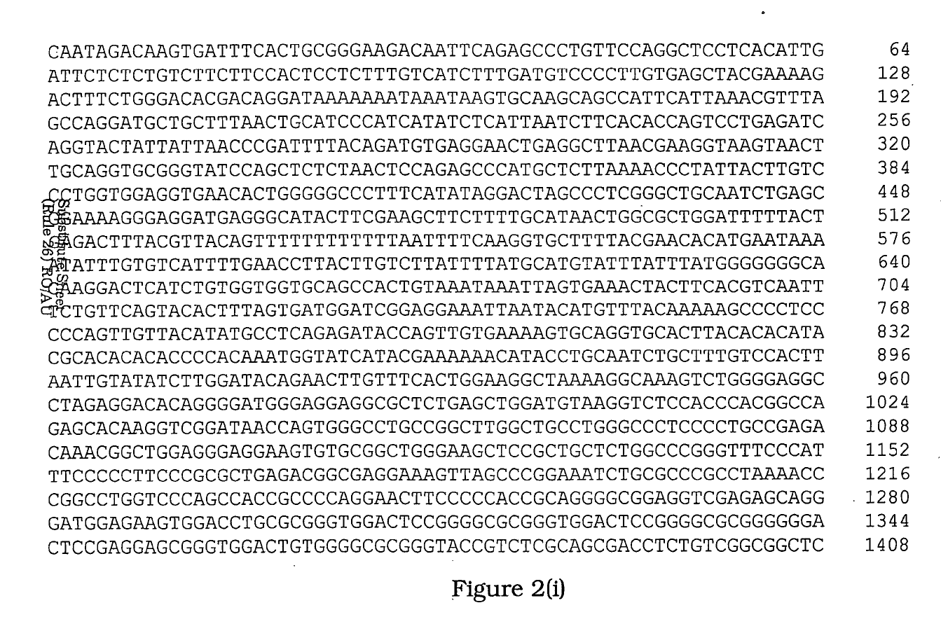

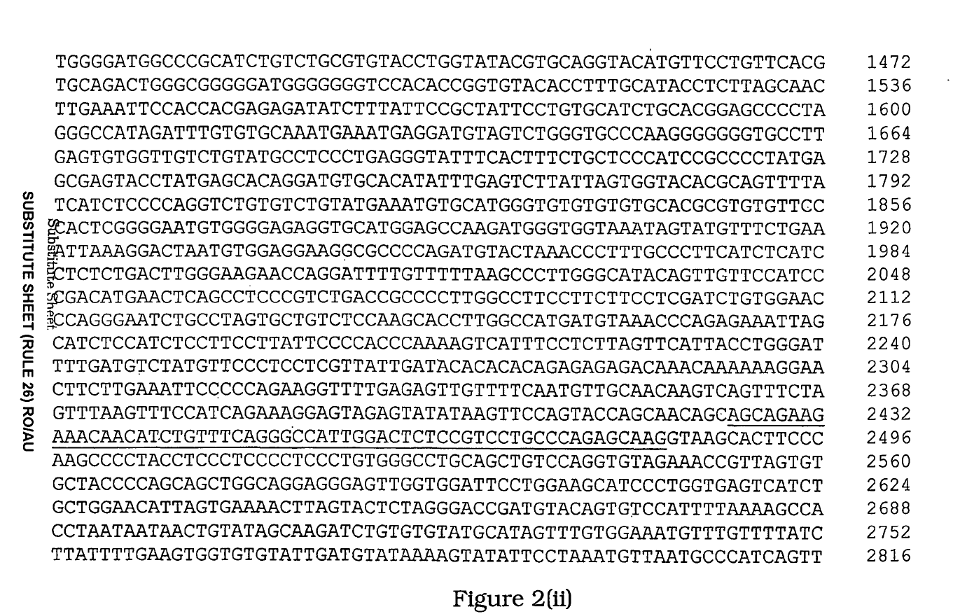

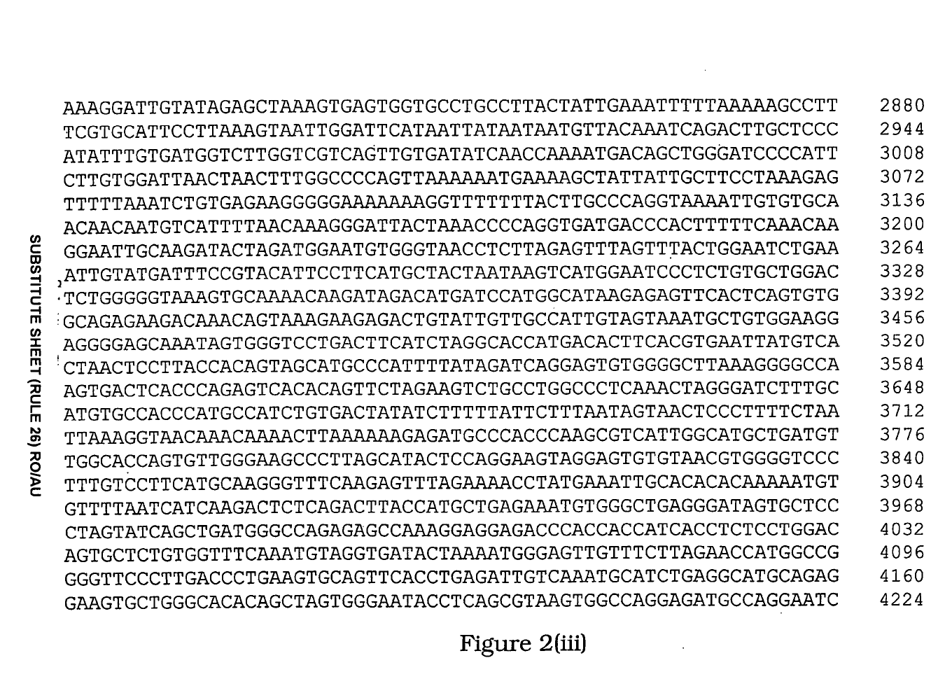

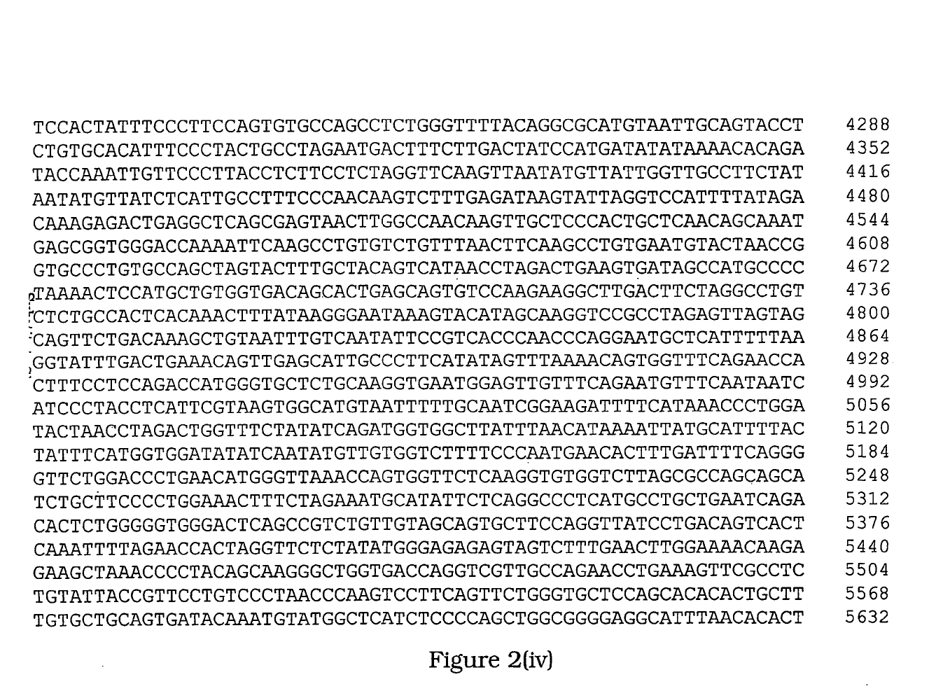

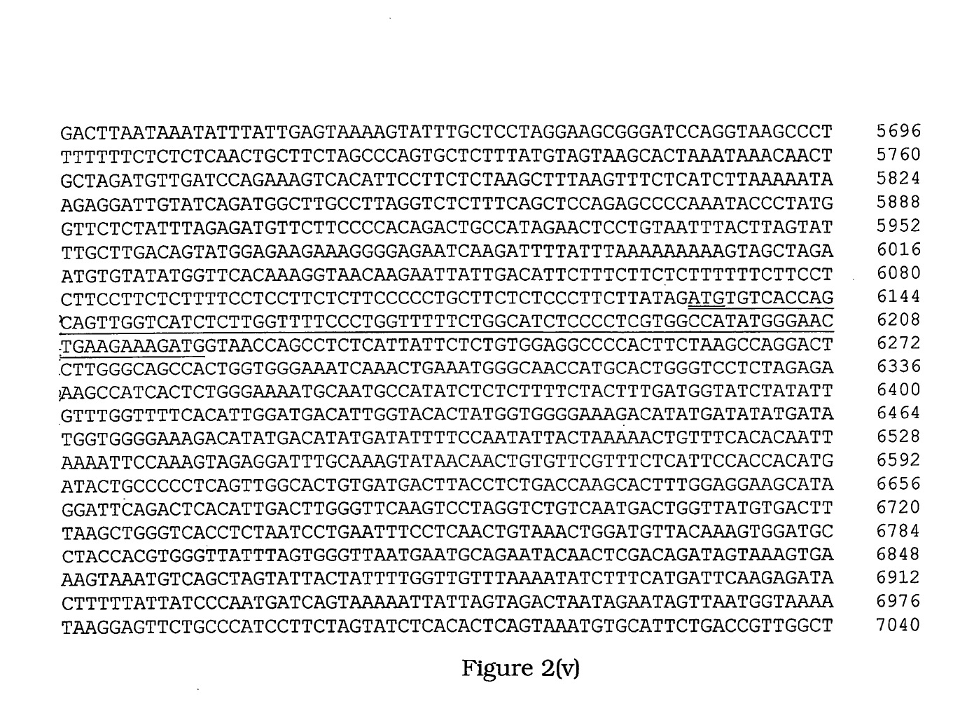

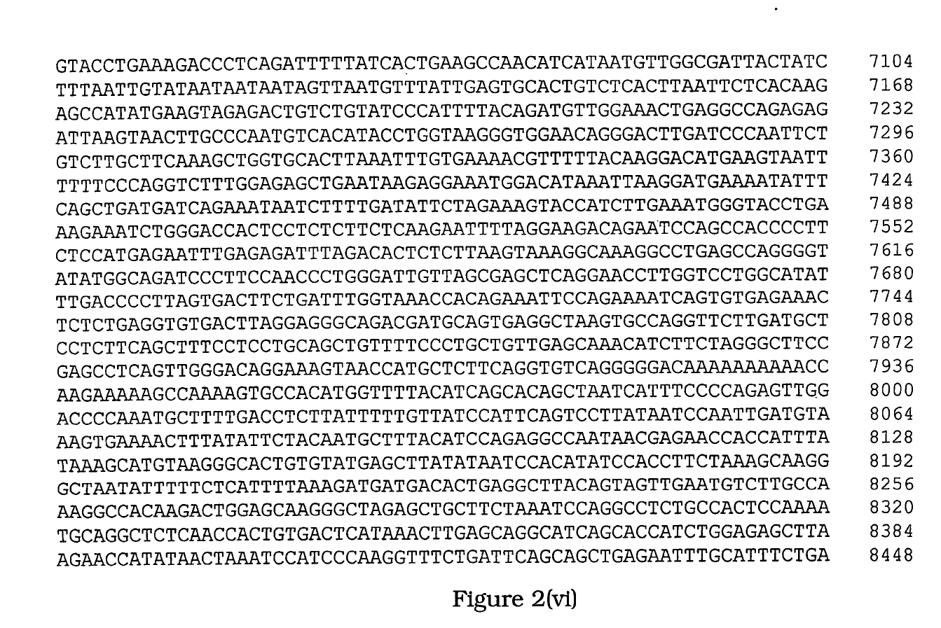

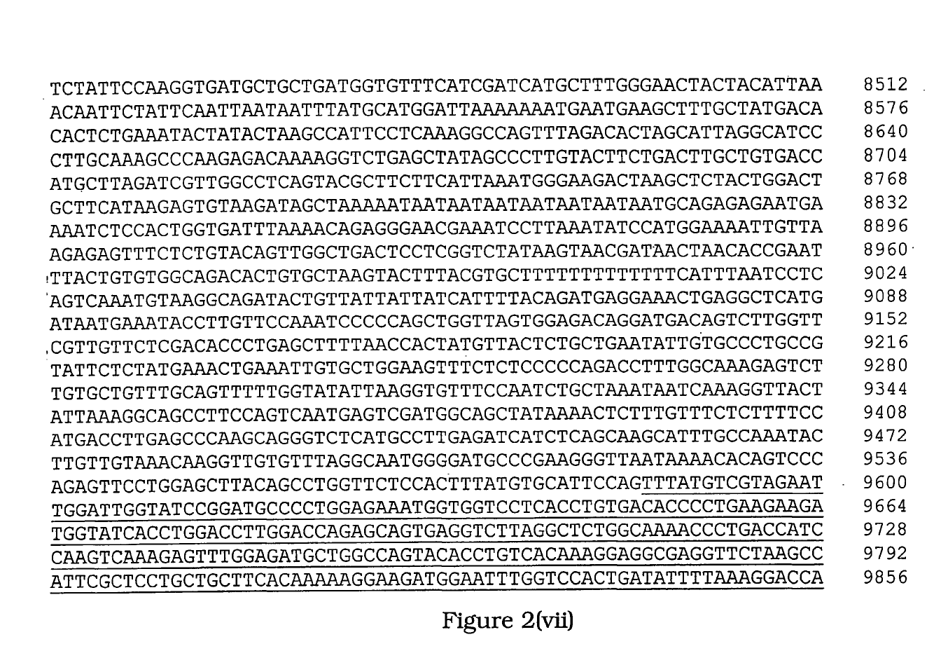

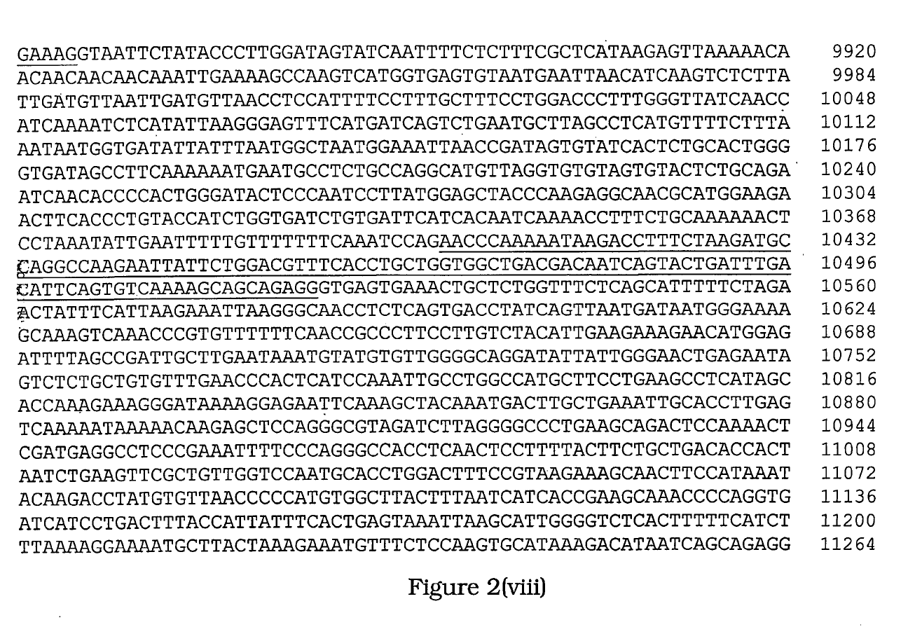

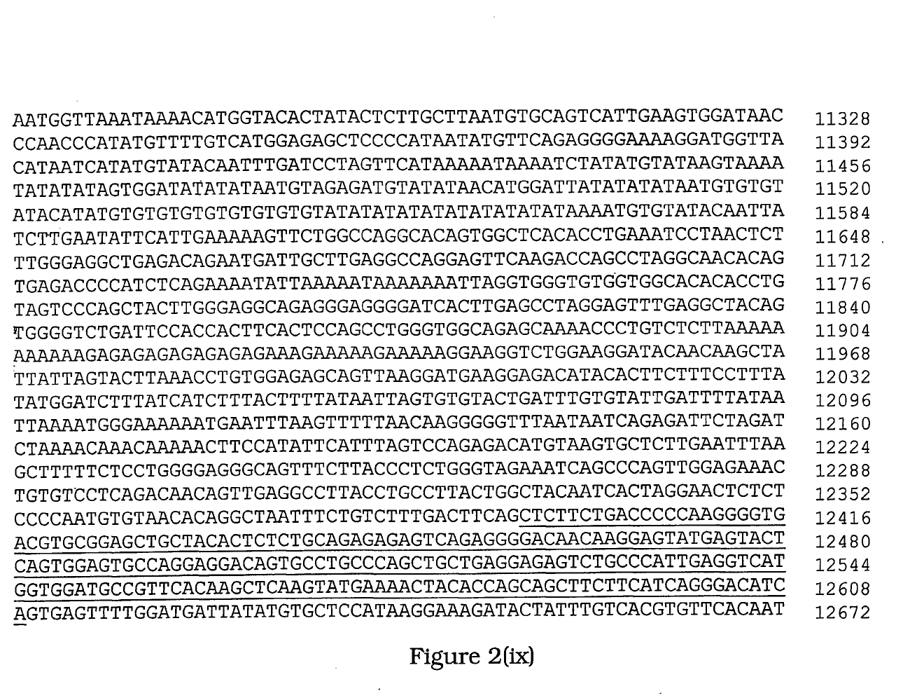

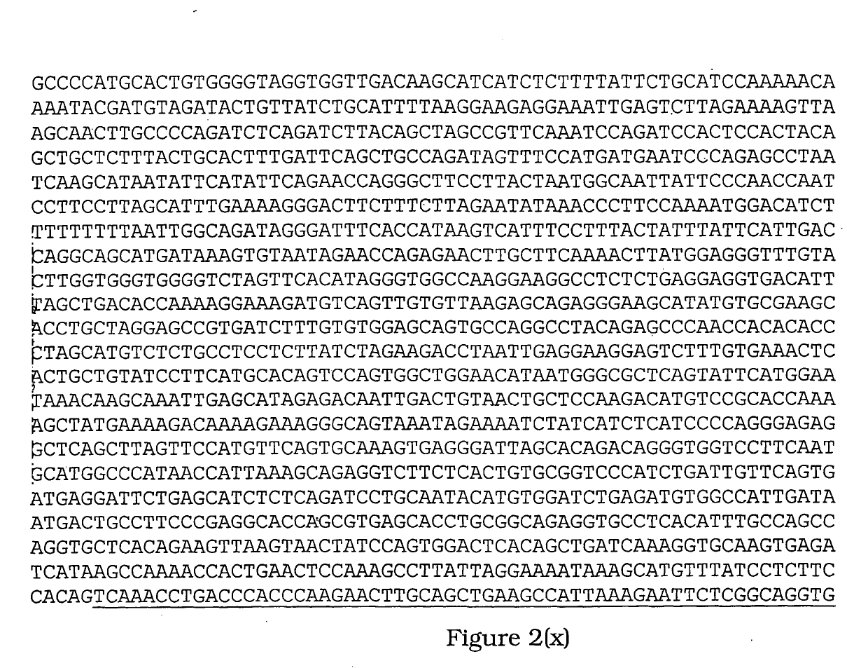

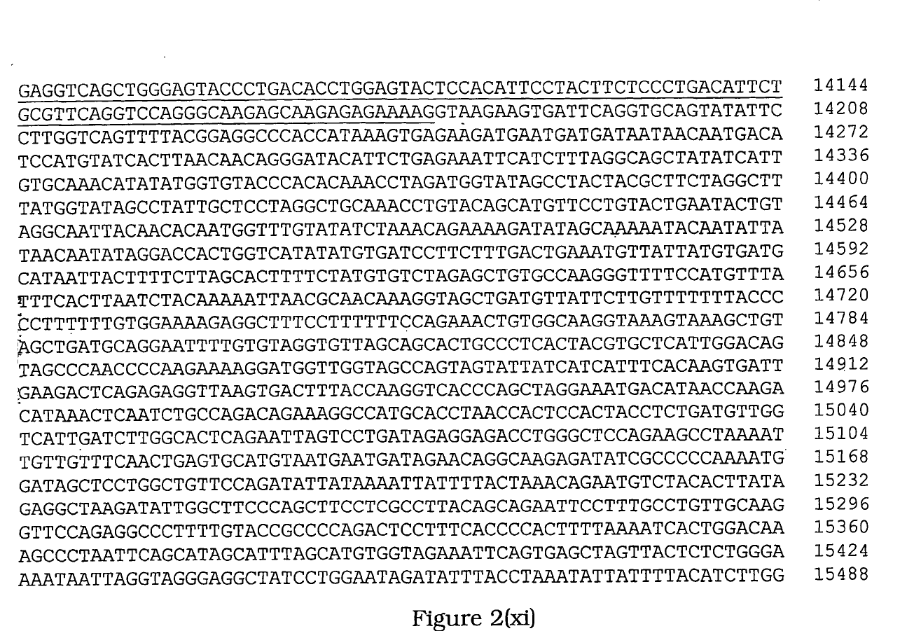

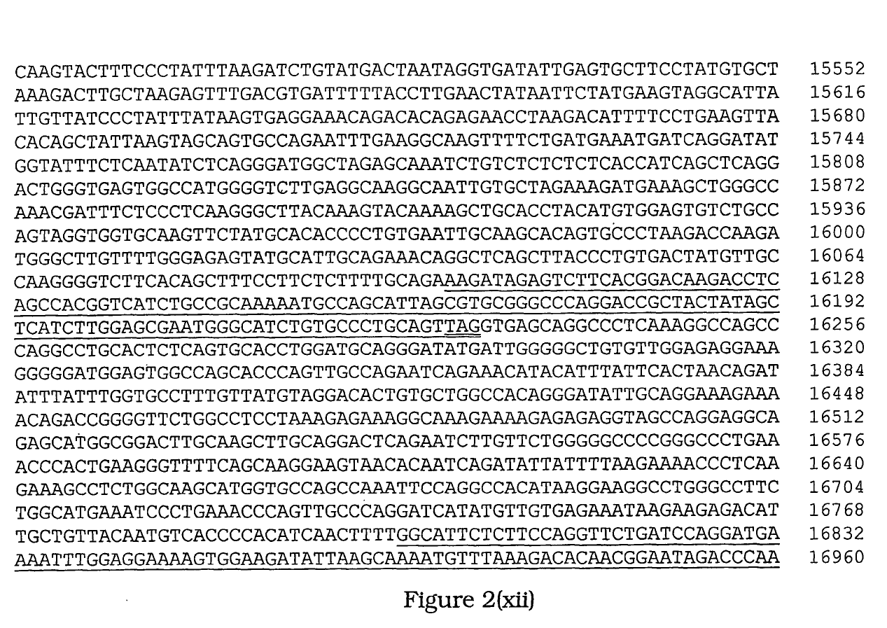

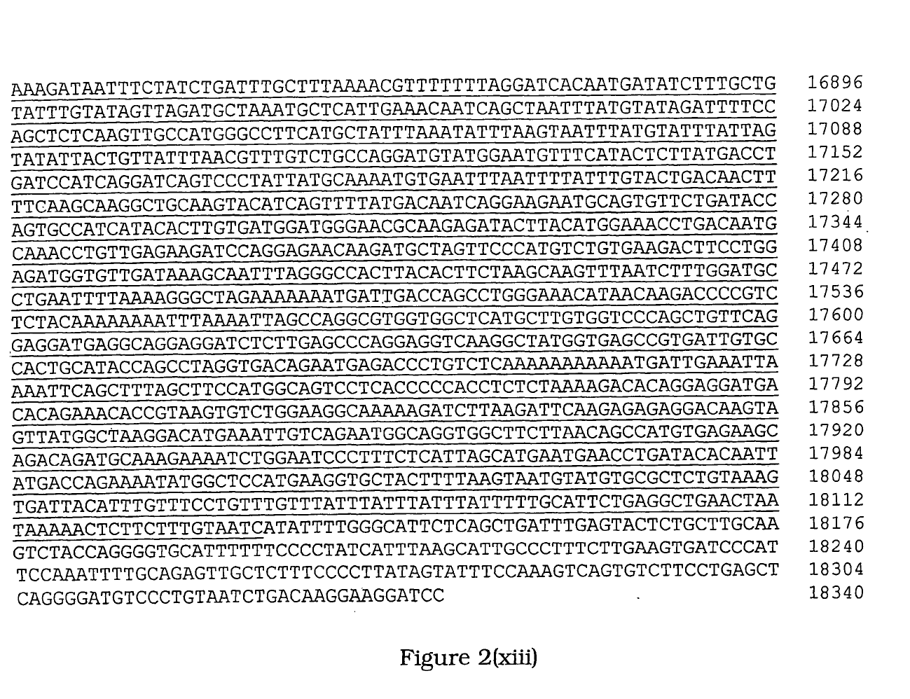

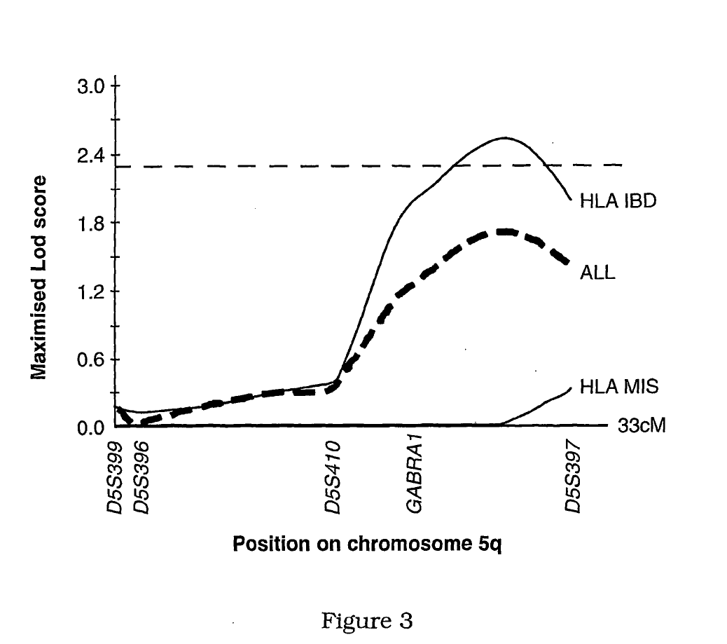

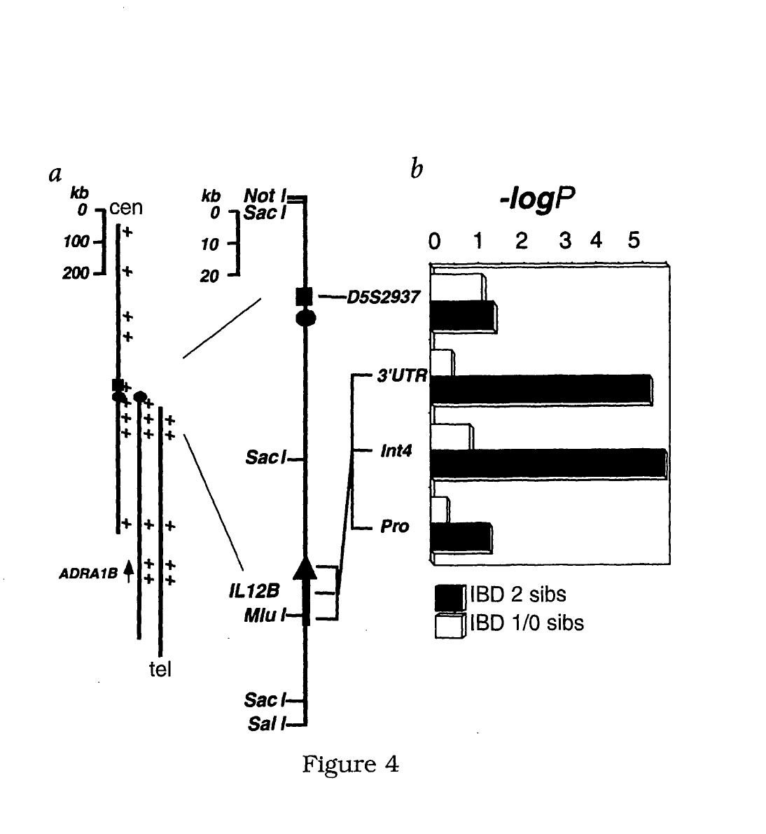

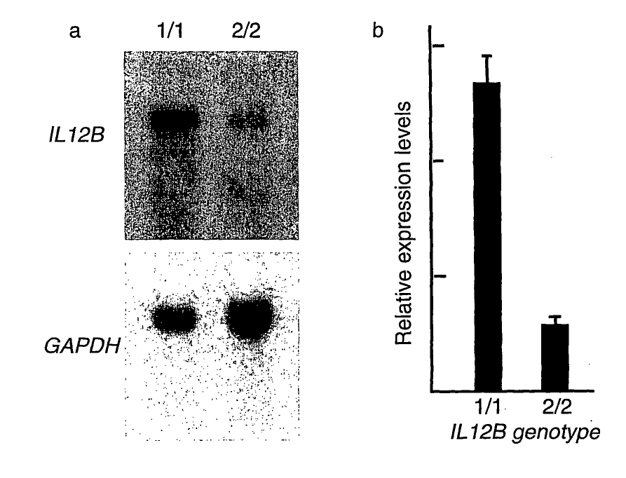

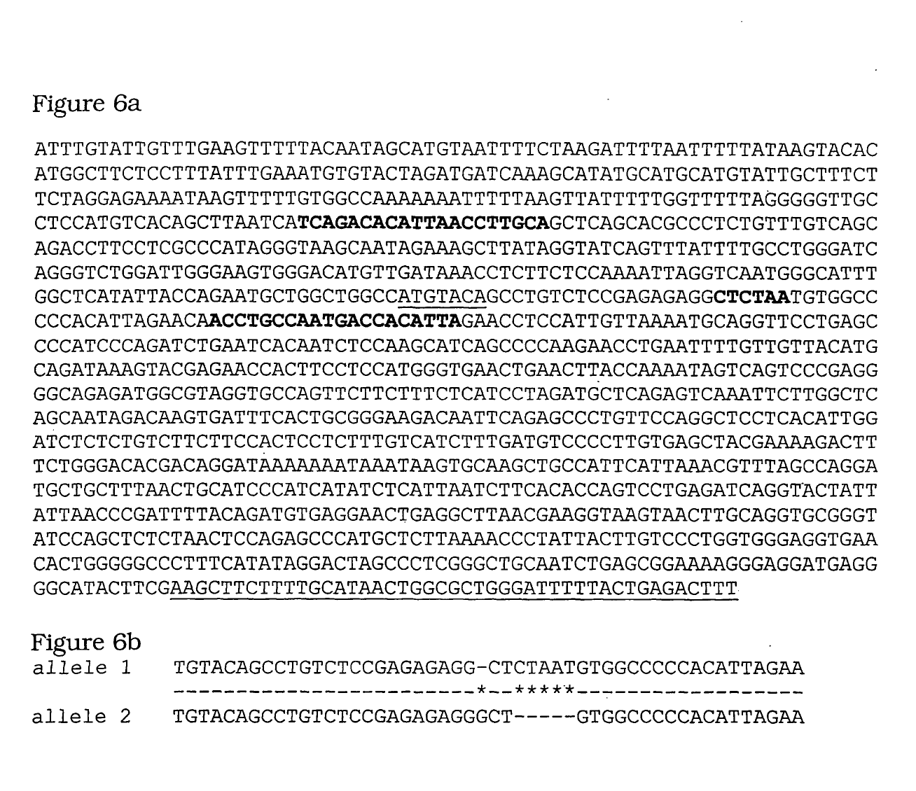

Although IL-12p40 had been mapped to chromosome 5q31-33 (Wairington et al., 1994), its position relative to microsatellite markers used in genetic studies has not been reported. Therefore, to localize IL-12p40 relative to other genes (eg GABRA1 (Johnson et al., 1992) and GABRA6 (Hicks et al., 1994)) and genetic markers D5S403, D5S10 and D5S412 9 in this region, radiation hybrid mapping was used (Boehnke et al., 1991). Comparison with previously mapped markers confirmed the assignment of IL-12p40 to distal chromosome 5q FIG. 1A). The optimal location for this gene was 3.3 centiRays (cR) telomeric from the microsatellite marker D5S412, and 3 cR centromeric from the anonymous DNA sequence, WI-9929 and a further 2.2 cR from D5S403 (FIG. 1A). These results integrating the genetic (Weissenbach et al., 1992) and physical maps of distal chromosome 5q will be useful for further genetic studies examining potential roles for IL-12p40 in disease. An inspection of the map of distal human chromosome 5 does not reveal any known diseases mapping to this region which could be attributable to variants in IL-12p40.

A phage P1-derived artificial chromosome (PAC) clone was isolated from the human PAC library produced by (Ioannou et al., 1994) with primers designed to amplify a segment from the 3′ untranslated region of IL12p40. The PAC clone 93-1 had an insert size of 130 kb. The sequence from the SP6 end of this clone was used to design primers to isolate an overlapping clone from a bacterial artificial chromosome (BAC) library (Osoegawa et al., 1998). A high resolution map of these clones is shown in FIG. 1B. Characterisation of these clones showed that they both contained the complete gene which also was arranged 3′->5′ with respect to the centromere. The MluI site within the IL-12p40 promoter was located 90 kb from the SP6 end of the PAC clone (FIG. 1B).

Determination of IL-12p40 Genomic Sequence

Only part of the promoter and the exon 1 genomic sequences of human IL-12p40 have been determined previously (Ma et al., 1996). In order to complete the sequence of the human IL-12p40 gene, the PAC clone, 93-1, was used as a template. DNA sequencing was performed using a walking strategy with fluorochrome-labeled dideoxynucleotide terminators, followed by automated analysis. This strategy was used because the priming oligonucleotides could also be used to generate PCR products for polymorphism testing.

A total of over 18 kb of sequence was determined and is deposited with Genbank (FIG. 2 provides the complete genomic sequence of the IL-12p40 gene). By alignment of this genomic sequence with the previously published cDNA sequences (Gubler et al., 1991; Wolf et al., 1991) the exact location and sequences of the exons were defined. Sequences at the exon-intron boundaries are reported in Table 7. The organization of the IL-12p40 gene is shown schematically in FIG. 1C. An unusual feature of the gene is that it has untranslated exons at both its 5′ and 3′ ends. Translation from its corresponding mRNA would be initiated at the first codon in exon 2 and would terminate at the last codon of exon 7.

Comparison of Genomic and cDNA Sequences

Sequence differences were observed between the sequence of the PAC clone that was determined and the previously reported IL-12p40 sequences, as shown in Table 8. These sequence differences could either represent genetic polymorphisms or arise from sequencing errors. In order to test whether the differences between the PAC sequence and the cDNA sequences were representative of alleles of the IL-12p40 gene, primers were designed to amplify the relevant regions of the gene from genomic DNA of different individuals (anonymous donors of European descent). PCR products were tested for the presence of genetic variants by either restriction enzyme digestion (where appropriate) or by direct sequencing. In this way, the A->C change in the 3UIR, resulting in creation of a TaqI site, was defined as a true genetic variant. In contrast, the C->G change resulting in the K->N amino acid substitution (exemplified by sequence HUMNKSFP40 (Wolf et al., 1991)) could not be found in DNA representing 224 chromosomes, including 97 which had the same IL12p40 allele as HUMNKSFP40, as defined by the presence of the 3UTR TaqI (not shown). Similarly, neither the exon 7 difference nor the promoter differences between the PAC and the published sequences could be confirmed.

Further Search for polymorphisms in the IL-12 p40 Gene

DNA from different individuals representative of the TaqI− and TaqI+ alleles was tested for further differences in and around the IL-12p40 gene. Polymorphisms were sought by PCR amplification followed by SSCP, restriction enzyme digestion or direct sequencing. Variants found by SSCP were confirmed by subsequent sequencing (or other methods as appropriate) of samples from a number of unrelated individuals. The results are summarized in Tables 9 and 10. Our major interest was in finding whether commonly occurring IL-12p40 polymorphisms exist as these may be useful for testing in various disease situations. It should be noted that the possibility of other, rarer, IL12p40 variants was not tested and is not excluded.

Despite extensive searching, no coding region sequence differences were found. Simple sequence repeat polymorphisms were discovered in introns 2 and 4. However, these had limited heterogeneity, with only two and three alleles found for each, respectively. A number of apparent single nucleotide substitutions were found. All the polymorphisms listed in Table 9 and 10 were confirmed by sequencing. Some polymorphisms were examined further in a large sample of unrelated individuals of diverse European descent. In particular, the 3′UTR alleles were found to be in Hardy-Weinberg equilibrium, with the TaqI− allele having a frequency of 0.82. The TA repeat polymorphism in intron 4 showed a similar distribution, also in Hardy-Weinberg equilibrium. The longest allele of this polymorphism is probably in linkage disequilibrium with the 3′UTR TaqI+allele. Even though the sample size was small, there was a suggestion that other polymorphisms may not be in linkage disequilibrium, notably the two single base changes, each A->G, within 14 bp in intron 2 (Table 9, 10). In sequencing this region from 8 individuals, 2 were heterozygous for either form, while of the homozygotes, 5 had the AA haplotype and 1 each the AG and GG haplotypes.

There was clustering of the DNA sequence variations. Of the twelve polymorphisms found, four were in intron 1, three were in intron 2, and two in intron 4. The changes in introns 1 and 2 appeared in pairs separated by no more than 60 bp. No polymorphisms were found (by SSCP analysis) in introns 5 and 6. In searching for genetic variants in 1L-12p40, no changes were found which would give rise to amino acid changes. An apparent amino acid substitution in comparison with a previously described cDNA sequence could not be found in testing an additional 128 chromosomes. The dearth of any coding sequence changes indicates a high level of conservation between the human subjects tested. Perhaps it was not surprising that no sequence variants were found that resulted in amino acid substitutions, given that IL-12p40 plays a fundamental role in immune regulation (Trinchieri G., 1995). However, this contrasts with the large number of differences displayed between species: there are 116 differences in amino acid sequences between the approximately 335 residues of the mouse and human IL-12p40 proteins (Gubler et al., 1991; Wolf et al., 1991; Tone et al., 1996). Despite the sequence differences, the genomic organization of mouse (Wolf et al., 1991) and human IL-12p40 genes is similar: both have 8 exons and the relative size of the introns is similar in both species. The mouse gene has an untranslated first exon but, unlike the human gene, the last exon does encode part of the final protein product (Wolf et al., 1991).

The polymorphisms described are useful in genetic studies to determine the role of IL-12p40 in regulation of the immune response in health and disease.

Method

Polymorphisms in the IL12p40 gene were sought. DNA fragments covering the entire gene were amplified from a panel of up to 27 unrelated donors and S was performed as follows. Forward and reverse primers shown were selected from the PAC sequence, and used to amplify specific segments of the IL-1 gene. “Standard” PCR conditions were performed incorporating 32 P-dATP: 2′ at 95° C. followed by 35 cycles of 20 s at 95° C., 20 s at 55° C., and 30 s at different extension times are indicated. For SSCP, a portion (1 ml) of the reaction mix was added to 5 ml of loading buffer (95% formamide, 20 mM EDTA, NaOH, 0.05% bromophenol blue and 0.15% xylene cyanol) heated at 90° C. for 1 min. and loaded onto a 4 to 5% polyacrylamide gel (1:45 or 1:90 ratio of N-methylene bisacrylamide to acrylamide). The intron 7 product was digested with EcoRV and HindIII prior to SSCP. Electrophoresis was performed overnight at room temperature. The gel was blotted on filter paper and exposed to autoradiography overnight at −70° C. Fragments which gave variable products were selected for sequencing. Note: the sequencing panel was selected so as to be enriched for individuals homozygous for the exon 8 TaqI allele number of individuals sequenced who were homozygous for the canonical PAC sequence, or for the alternate non-PAC sequence are shown, as is the number heterozygotes.

### EXAMPLE 7

### Linkage Disequilibrium of Regulatory IL12P40 Alleles with Type I Diabetes

Results

To test whether IL12B may be a susceptibility gene in human T1D, 249 sibpairs were typed for markers on chromosome 5q33-34, to which IL12B was mapped (Warrington et al., 1994). Testing multiplex families for markers from this region initially resulted in a modest lod score, suggestive of linkage to a susceptibility gene (FIG. 3). Stratification of sibpairs has proven useful in revealing linkage in multipoint analyses, allowing clear definition of the susceptibility locus IDDM13 (Morahan et al., 1996; Fu et al., 1998; Larsen et al., 1999). Applying stratification to the 5q data revealed a difference between sibpairs sharing HLA haplotypes and those differing at HLA (FIG. 3). The HLA-identical sibpairs showed linkage to this region with a maximized lod greater than 2.3; this susceptibility locus is provisionally amed IDDM18. In contrast, and unlike the case for IDDM13 (Morahan et al., 1996), there was no evidence of linkage in the HLA mismatched sibs. This emphasizes the genetic heterogeneity of TID, such that different subgroups have susceptibility arising from different interactions of HLA and non-HLA genes.

The linkage analyses indicated that IDDM18 may reside near IL12B. The complete sequence of, and genetic polymorphisms in and around, IL12B have been described (Huang et al., in press). Although there were no common coding region variants, we found useful polymorphisms in the 3′ UTR, intron 4 and the promoter. These polymorphisms were typed and the transmission disequilibrium test (Spielman et al., 1993) TDT was applied. There was significant excess transmission of particular intron 4 and 3′ UTR alleles, but not of alleles defined by the promoter polymorphism (Table 13). (The intron 4 and 3′ UTR alleles are in linkage disequilibrium, so further discussion is limited to the latter). A physical map of >1 Mb surrounding IL12B was constructed (FIG. 4a) and searched for futher downstream polymorphisms; one resulting marker, D5S2937, has 10 alleles, none of which singly or jointly generated significant TDT results (Table 13). These observations were confirmed using the Tₛₚ statistic, which adjusts for testing more than one affected subject per family (Martin et al., 1998) (Table 13). Similar results were also obtained testing only one affected sib per family (data not shown).

To further test the involvement of IL12B polymorphisms in T1D, the families were divided into two groups: one showing linkage to 5q33-34 (which would be expected to shown the influence of the disease allele) and one which did not (and would therefore predominantly include sibs who had TID due to other susceptibility loci). The TDT was applied to each group (FIG. 4b). Evidence for preferential transmission increased in the linked group, whereas there was no significant deviation in transmission of alleles to the unlinked group. (Although this method of selection of sibs for linkage will affect matching of alleles within families, it should not affect genotypes between families, and hence should not affect the overall TDT. In fact, similar results were obtained when the analysis was restricted to the first affected sib (data not shown). The results showed preferential transmission in only those families in which T1D was linked to IL12B, indicating that TID is mediated in part by the IL-12-linked causative polymorphism. There was again preferential transmission of the 3′ UTR polymorphism, but none at the promoter polymorphism only 20 kb upstream (FIGS. 4a,b).

If the 3′ UTR polymorphism itself contributes to susceptibility, the offspring of homozygous parents should not show linkage, unlike offspring of heterozygous parents (Robinson et al., 1993). Because the frequency of the susceptibility allele is 0.8, the families in which at least one parent is homozygous will be in the majority, helping to explain the low lod scores obtained in the original analysis. Essentially, all the evidence for linkage (MLS=2.632) was maintained in the group with at least one heterozygous parent; there was no evidence for linkage to IL12B promoter alleles in families in which both parents were homozygous at the 3′ UTR (MLS=0.388).

It was crucial to confirm the above findings of preferential transmission of IL12B 3′ UTR allele 1 to T1D subjects. The Australian IDDM DNA Repository has been established and into which 238 families have been recruited and typed for IL12B-associated polymorphisms. The results confirm those obtained above: preferential transmission of allele 1 of the 3′ UTR polymorphism, and lack of bias in transmission of promoter alleles (Table 14). The Australian IDDM DNA Repository families were also typed at a novel polymorphism, DS2340, located 12 kb downstream of the IL12B 3′ UTR. This marker did not yield significant TDT results (Table 14). Combining the results from both TDT analyses of the IL12B 3′ UTR, the null hypothesis of lack of association of the IL12B 3′ UTR with TID may be rejected (overall P=3.5×10⁷). Significant linkage disequilibrium appears confined to a region of approximately 30 kb in which IL12B is the only known gene.

The results show that the 3′ UTR allele 1 is preferentially transmitted to T1D subjects, and hence either itself confers susceptibility or is in linkage disequilibrium with the disease-predisposing variant; allele 2 is preferentially non-transmitted, so it may be associated with T1D resistance. As no common change was found in its coding sequences, if IL12B is involved in TID susceptibility then its alleles should show some other functional difference. To address this, EBV-transformed cell lines (which are known to express IL-12; refs. Wolf et al., 1991; Gubler et al., 1991) homozygous for each allele were identified. Expression of IL12B was significantly reduced in the 2/2 genotype cell line relative to the 1/1 line (FIG. 5). The 3′ UTR polymorphism is located over 1 kb from the mRNA degradation element (Zubiaga et al., 1995), so it is unlikely that the observed difference between the cell lines is due to differences in stability. The inference that the 3′ UTR polymorphism may affect gene expression is supported by a similar finding for the rat gene spi2.3 (LeCam et al., 1995). If differences in IL12B expression result in different levels of protein, then individuals with the susceptibility allele should produce more IL12p40. Higher IL-12 levels were found in relatives of T1D probands (Szelachowska et al., 1997). Increased IL-12 may promote Th1 cells, and aggravate autoimmune destruction of β-cells, causing T1D (as in NOD mice (Katz et al., 1995; Trembleau et al., 1995)). In contrast, lower levels of IL-12 should reduce susceptibility because IL-12 antagonists can protect NOD mice from diabetes (Trembleau et al., 1997).

Materials and Methods

Genotyping

We tested a total of 249 affected sibpairs, including families that were previously described (Morahan et al., 1996) and an additional 120 families obtained from the British Diabetes Association. An additional independent cohort of 235 predominantly simplex families was also recruited into the Australian IDDM DNA Repository. DNA from individuals from multiplex families were typed using either anonymous microsatellite markers (Weissenbach et al., 1992) or the highly polymorphic repeat within GABRA1 (Johnson et al., 1992). We tested polymorphisms in and around IL12B as described (Huang et al., in press). The D5S2937 marker is a simple sequence repeat which was generated from inspection of the draft sequence of the BAC 9p16 from 5q33-34 obtained from the DOE's Joint Genome Institute (ftp://ft]2.1gi-psf.orgipub/JGI-data/Human/Ch5/Draft/). primers to amplify this TAA repeat were 5′-GGGTAAGCGATTCAAA-CATT-3′ (<400>137) (forward) and 5′GGTATTGCATTGTAGGCACAT-3′ (<400>138) (reverse). D5S2940 is a C(T)n repeat located 12 kb centromeric of the 3′ UTR and was amplified with primers 5′GGGCAACAAGAGTGAAACT-3′ (<400>139) and 5′-TCAAAAGAGGTCCGTCTAAA-3′ (<400>140).

Genetic Analyses

We carried out multipoint linkage analysis using the MAPMAKER/Sibs software program (Kruglyak et al., 1995), and TDT analyses (Spielman et al., 1993) using both the GAS software package (Young, A., 1994) and Tsp program (Martin et al., 1998).

Gene Expression

We typed EBV-transformed cell lines from the 4th Asia-Oceania Histocompatibility Workshop cell line panel Degli-Esposti et al., 1993) for the IL12B 3′ UTR allele, and cell lines representing the 1/1 and 2/2 genotypes were selected. We isolated total RNA from these cell lines using guanadinium thiocyanate and purified it by CsCl-density gradient centrifugation. Northern-blot analysis was performed by standard methods (Sambrook et al., 1989) with human IL12B and GAPDH cDNA probes. The levels of IL12B MRNA in each cell line relative to GAPDH was determined by densitometry in three separate experiments. Similar results were obtained by RT-PCR (data not shown).

### EXAMPLE 8

### Polymorphisms Haplotypes as a Marker of Disease Susceptibility or Resistance

The combination of particular polymorphisms is used to define IL12B haplotypes. These haplotypes are used to test for susceptibility or resistance to immune related diseases in which IL12 production and/or Th1-Th2 regulation may be relevant.

An example of the use of such haplotypes is demonstrated in the table below. Combining promoter and 3′ UTR alleles generates 4 haplotypes. The appearance of these haplotypes may be compared between different groups which differ in a relevant phenotype. To illustrate this point, consider subjects with diabetes and first degree relatives who do not have diabetes but who have autoantibodies (i.e. “preclinical”). The table shows that there is a difference in the proportion of haplotype C homozygous individuals in these groups. Thus haplotypes may be used to predict likelihood to proceed from early autoimmunity to diabetes. Haplotypes may be used in this way to test for susceptibility or resistance to other disease conditions or predisposition to mounting Th1 or Th2 type immune responses.

Haplotype analysis of IDDM and preclinical subjects.

| Subjects | Haplotype C/C | Other haplotypes | P |
|---|---|---|---|
| IDDM | 122 | 124 | 0.005 |
| Preclinical | 9 | 32 | |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 7
Sequences of Exon-Intron boundaries.

| | |
|---|---|
| Intron | Sequence |

| | | |
|---|---|---|
| 1 <400>116 | GCCCAGAGCAAGGTAAGCACTTCC . . . CCCTTCTTATAGATG TGTCACCAG | <400>117 |
| 2 <400>118 | TGAAGAAAGATGGTAACCAGCCTC . . . TGTGCATTCCAGTTTATGTCGTAG | <400>119 |
| 3 <400>120 | AGGACCAGAAAGGTAATTCTATAC . . . TTTCAAATCCAGAACCCAAAAATA | <400>121 |
| 4 <400>122 | AAGCAGCAGAGGGTGAGTGAAACT . . . CTTTGACTTCAGCTCTTCTGACCC | <400>123 |
| 5 <400>124 | TCAGGGACATCAGTGAGTTTTGGA . . . CCTCTTCCACAGTCAAACCTGACC | <400>125 |
| 6 <400>126 | AAGAGAGAAAAGGTAAGAAGTGAT . . . TCTCTTTTGCAGAAAGATAGAGTC | <400>127 |
| 7 <400>128 | CCCTGCAGT TAGGTGAGCAGGCCC . . . ATTCTCTTCCAGGTTCTGATCCAG | <400>129 |

Sequences of Exon-Intron boundaries. The complete IL12p40 genomic sequence was determined using PAC 93.1 as a template. The sequence has been deposited in Genbank. Exon-intron boundaries were determined by comparison with published CDNA sequences (Gubler et al., 1991; Wolf et al., 1991). Exon sequences are shown in bold; splice donor and acceptor sites are single underlined. The start and stop codons (double underlined) are the first and last codons in exons 2 and 7 respectively. Sizes of introns are indicated in FIG. 1C.

TABLE 8
Comparison of genomic and published IL12p40 sequences

| | | | | |
|---|---|---|---|---|
| Region | DNA source | Position | Sequence | Comment |

| | | | | | |
|---|---|---|---|---|---|
| Promoter | PAC 93-1 | 544 | <400>3 | AGTTTTTTTTTTTTAATTTTCAAGGTGCTT | |
| | | | | ----------------*------------- | No RE difference |
| | HSU89323 | | <400>4 | AGTTTTTTTTTTTTAAATTTCAAGGTGCTT | Could not confirm |
| Promoter | PAC 93-1 | 885 | <400>5 | AACATACCTGCAATCTGCTTTGTCCACTTA | |
| | | | | -----------------*------------ | No RE difference |
| | HSU89323 | | <400>6 | AACATACCTGCAATCTGATTTGTCCACTTA | Could not confirm |
| Promoter | PAC 93-1 | 1973 | <400>7 | CTAAACCCTTTGCCCTTCATCTCATCCTC | |
| | | | | --------------*-------------- | No RE difference |
| | HSU89323 | | <400>8 | CTAAACCCTTTGCC-TTCATCTCATCCTC | Could not confirm |
| Exon 6 | PAC 93-1 | 14041 | <400>9 | TCAAACCTGACCCACCCAAGAACTTGCAGC | HUMCLMF40 = PAC |
| | | | | -------------------*---------- | Gives K -> N change |
| | HUMNKSFP40 | | <400>10 | TCAAACCTGACCCACCCAACAACTTGCAGC | Could not confirm |
| Exon 7 | PAC 93-1 | 16117 | <400>11 | AGATAGAGTCTTCACGGACAAGACCTCAGC | HUMCLMF40 = PAC |
| | | | | ---------------*-------------- | Silent change |
| | HUMNKSFP40 | | <400>12 | AGATAGAGTCTTCACCGACAAGACCTCAGC | Could not confirm |
| 3′ UTR | PAC 93-1 | 16974 | <400>13 | TTGTATAGTTAGATGCTAAATGCT | L3 same; TaqI− |
| (exon 8) | | | | ----------*------------- | Creates a TaqI site |
| | HUMNKSFP40 | | <400>14 | TTGTATAGTTCGATGCTAAATGCT | L26 same; TaqI+ |

The IL12p40 genomic sequence summarized in FIG. 1C was compared with the previously published sequences of the promoter (HSU89323 Ma et al., 1996) and cDNAs(HUMCLMF40 (Gubler et al., 1991) and HUMNKSFP40 Wolf et al., 1991). Differences between these sequences are shown. These differences may affect amino acidsubstitutions or restriction enzyme (RE) recognition sequences as
oted. Differences were confirmed by sequencing of independently derived DNA samples. L3 and L26 are DNA samples derived from anonymous donors homozygous for the 3′UTR TaqI − and TaqI + alleles, respectively.

TABLE 9
Detection and Confirmation of IL12p40 polymorphisms

| | |
|---|---|
| Product | Reaction |

| | | | |
|---|---|---|---|
| Polymorphism | Primers (5′->3′) | size | conditions* |

| | | | |
|---|---|---|---|
| Intron 1 3696 | GGCTTAAAGGGGCCAAGT <400>15 | 401 | Standard |
| | AGGGAGCACTATCCCTCAGC <400>16 | | |
| Intron 1 3757 | GGCTAAAGGGGCCAAGT <400>17 | 401 | Standard |
| | AGGGAGCACTATCCCTCAGC <400>18 | | |
| Intron 1 4572 | ATGTTATCTCATTGCCTTC <400>19 | 511-513 | Standard |
| | AAGTGGTTCTGAAACCACTG <400>20 | | |
| Intron 1 4793 | ATGTTATCTCATTGCCTTTC <400>21 | 511-513 | Standard |
| | AAGTGGTTCTGAAACCACTG <400>22 | | |
| Intron 2 8798 | GGGAAGACTAAGCTCTACTG <400>23 | 128-131 | Standard |
| | GGATTTCGTTCCCTCTGTTT <400>24 | | |
| Intron 2 8930 | GGGAAGACTAAGCTCTACTG <400>25 | 413 | Standard |
| | CAACGAACCAAGACTGTCAT <400>26 | | |
| Intron 2 8944 | GGGAAGACTAAGCTCTACTG <400>27 | 413 | Standard |
| | CAACGAACCAAGACTGTCAT <400>28 | | |
| Intron 3 9910 | TTCTAAGCCATTCGCTCCTG <400>29 | 188 | Standard |
| | GTTAATTCATTACACTCACC <400>30 | | |
| Intron 4 11244 | TACTTCTGCTGACACCACTA <400>31 | 436 | Standard |
| | GAACTAGGATCAAATTGTATAC <400>32 | | |
| Intron 4 11563 | GGTTACATAATCATATGTA <400>33 | 254-258 | Standard |
| | GTTAGGATTTCAGGTGTGAG <400>34 | | |
| Intron 7 16521 | TAGCTCATCTTGGAGCGAAT <400>35 | 982 | 70 s at 72° C. |
| | AACATTCCATACATCCTGGC <400>36 | | |
| Exon 7 16117 | GAAAGGCCATGCACCTAAC <400>37 | 1,285 | 90 s at 72° C. |
| | TCCAGGTGCACTGAGAGT <400>38 | | |
| Exon 8 16974 | TTTGGAGGAAAAGTGGAAGA <400>39 | 300 | 2′ final at 72° C. |
| | AACATTCCATACATCCTGGC <400>40 | | TaqI digestion |

| | | | | |
|---|---|---|---|---|
| Detection | Number | Number | Number homozygous for | Number |

| | | | | | |
|---|---|---|---|---|---|
| Method | screened | sequenced | PAC allele | non-PAC allele | Heterozygous |

| | | | | | |
|---|---|---|---|---|---|
| SSCP | 23 | 16 | 1 | 11 | 4 |
| SSCP | 23 | 16 | 3 | 10 | 3 |
| SSCP | 27 | 15 | 1 | 12 | 2 |
| SSCP | 27 | 13 | 2 | 10 | 1 |
| SSCP | 27 | 6 | 4 | 2 | 0 |
| SSCP | 27 | 9 | 1 | 7 | 1 |
| SSCP | 27 | 8 | 2 | 5 | 1 |
| SSCP | 27 | 9 | 3 | 5 | 1 |
| SSCP | 24 | 14 | 2 | 12 | 0 |
| SSCP | see Table 5 | | | | |
| EcoRV, HindIII | | 15 | 1 | 14 | 0 |
| digest, SSCP | | | | | |
| direct sequence | | 9 | 9 | 0 | 0 |
| | see Table 5 | | | | |

TABLE 10
Allelic differences in IL12p40 sequences.

| | | | | |
|---|---|---|---|---|
| Intron | DNA | Position | Sequence | No. Alleles |

| | | | | |
|---|---|---|---|---|
| 1 | L26 | | TCTTTAATAATAACTCCCTTTT | <400>41 |
| | | | ---------*------------ | |
| | PAC | 3696 | TCTTTAATAGTAACTCCCTTTT | <400>42 |
| | L26 | | GCCCACCCAAGTGTCATTGG | <400>43 |
| | | | -----------*-------- | |
| | PAC | 3757 | GCCCACCCAAGCGTCATTGG | <400>44 |
| | L26 | | TCAAGCCTG-TCTGTTTAA | <400>45 |
| | | | ---------**--------- | |
| | PAC | 4572-3 | TCAAGCCTGTGTCTGTTTAA | <400>46 |
| | L26 | | CCGCCTAGACTTAGTAG | <400>47 |
| | | | ---------*------- | |
| | PAC | 4793 | CCGCCTAGAGTTAGTAG | <400>48 |
| 2 | L26 | | TAAAAATAATAATAATAATAATAATAATAATG | <400>49 2 |
| | | | ------***----------------------- | 2 |
| | PAC | 8798-8800 | TAAAAA---TAATAATAATAATAATAATAATG | <400>50 |
| | L26 | | CTCCTCAGTCTATAAGTAACAATAACTA | <400>51 |
| | | | ------*-------------*------- | |
| | PAC | 8930; 8944 | CTCCTCGGTCTATAAGTAACGATAACTA | <400>52 |
| 3 | L26 | | CGCTCATAAGGGTTAAAAACAACAACAAC | <400>53 |
| | | | ----------*------------------ | |
| | PAC | 9910 | CGCTCATAAGAGTTAAAAACAACAACAAC | <400>54 |
| 4 | L26 | | TCTCCAAGTGCAAAAAGACATAATCAGCAG | <400>55 |
| | | | ------------*----------------- | |
| | PAC | 11244 | TCTCCAAGTGCATAAAGACATAATCAGCAG | <400>56 |
| | L26 | | TATATATATAT----AAAATGTGTATACA | <400>57 3 |
| | | | -----------****-------------- | |
| | PAC | 11563-6 | TATATATATATATATAAAATGTGTATACA | <400>58 |
| 7 | L26 | | AGAGCATGGAGGACTTGCA | <400>59 |
| | | | ---------*--------- | |
| | PAC | 16521 | AGAGCATGGCGGACTTGCA | <400>60 |

Sequences are shown of polymorphisms between the PAC sequence andPCR products from a donor (L26) homozygous for IL12p40 3′UTR TaqI alleles. Geneticvariants were detected and confirmed by sequencing of products from other donors as described in Table 9. Position indicated is the position of the PAC sequence. The number of alleles observed for the repeat polymorphisms is indicated.

TABLE 11
Allele Frequencies of IL12p40 variants.

| | | | | | Hardy- |
|---|---|---|---|---|---|
| | | Frequency | | | Weinberg |
| Polymorphism | No. tested | Allele 1 | Allele 2 | Allele 3 | equilibrium |
| Intron 4 11563 | 336 | 0.79 | 0.2 | 0.01 | Yes |
| Exon 8 16974 | 382 | 0.82 | 0.18 | | Yes |

Allele frequencies were determined by genotyping unrelated subjects of diverse European descent. The intron 4 TA repeat polymorphism was detected on denaturing bis-acrylamide gels. The exon 8 TaqI allele was detected as follows: 2 ul of products digested with 1 unit of TaqI in a 1o ul reaction volume and incubate at 65° C. for 2 hours. The number of individuals with each genotype did not differ from that expected if the alleles were in Hardy-Weinberg equilibrium.

TABLE 12
Primers used for sequencing PAC93-1

| | |
|---|---|
| Forward primers: | Reverse primers: |

| | | | | | |
|---|---|---|---|---|---|
| <400>61 | PF | GGAAGGCGCCCCAGATGTA | P9R | GAATTGCATGCTGGGTTCTG | <400>86 |
| <400>62 | P3F | TCAGACACATTAACCTTGCA | P8R | CTGTGGCTTCCAGAGGTTAC | <400>87 |
| <400>63 | P4F | CAATAGACAAGTGATTTCACTG | P7R | TAATGTGGTCATTGGCAGGT | <400>88 |
| <400>64 | P6F | ATGCTTAATTATAACTATATTC | P5R | CAGATGAGTCCTTGTGCCCC | <400>89 |
| <400>65 | P7F | CCATAATAGGTTCATTGCCC | P4R | ACCCGGGCCAGAGCAGCG | <400>90 |
| <400>66 | Int1-3F | GGCTTAAAGGGGCCAAGT | P3R | GCGGAATAAAGATATCTCTC | <400>91 |
| <400>67 | Int1-6F | CCCACCACCATCACCTCT | P2R | GATGAGATGAAGGCAAAGG | <400>92 |
| <400>68 | Int1-5F | ATGTTATCTCATTGCCTTTC | PR | TCACCAGGGATGCTTCCAGG | <400>93 |
| <400>69 | Int1 (F) | GAAGAAAGGGGAGAATCAAG | Int1-5R | GGTTACCCACATTCCATC | <400>94 |
| <400>70 | Int2F | GGAAAATGCAATGCCATATC | Int1-2Fr | CTTATGCCATGGATCATGTC | <400>95 |
| <400>71 | Int2-1F | ATCCTGAATTTCCTCAACTG | Int1-7R | AAGTGGTTCTGAAACCACTG | <400>96 |
| <400>72 | Int2-2F | AGAGACTGTCTGTATCCCAT | Int1-4R | CCAGAGTGTCTGATTCAGC | <400>97 |
| <400>73 | Int2-4F | AGGCCTGAGCCAGGGGTAT | Int1-3R | AAGGCAAGCCATCTGATACA | <400>98 |
| <400>74 | Ex3F | TTCTAAGCCATTCGCTCCTG | Int2R | AGGCCAACGATCTAAGCATG | <400>99 |
| <400>75 | Int4F | ATTCTGGACGTTTCACCTGC | Int2-1R | GGAGTGGCAGAGGCCTGG | <400>100 |
| <400>76 | Int4F-4 | TACTTCTGCTGACACCACTA | Ex3-R | CACCATTTCTCCAGGGGCA | <400>101 |
| <400>77 | F-Int4.3 | GGATGAAGGAGACATACACT | Ex3-1R | CAACGAACCAAGACTGTCAT | <400>102 |
| <400>78 | IL-12A | GCCGTTCACAAGCTCAAGTA | Int3-1R | GTTAATTCATTACACTCACC | <400>103 |
| <400>79 | Int5F | GGACTTCTTTCTTAGAATAT | Int4-8R | ATAGGTCACTGAGAGGTTGC | <400>104 |
| <400>80 | IL-12E | ATCAAACCTGACCCACCCAA | Int4-3R | AGCTTGTTGTATCCTTCCAG | <400>105 |
| <400>81 | Int6F | ATGTGATCCTTCTTTGACTG | Int4R | TCATACTCCTTGTTGTCCCC | <400>106 |
| <400>82 | Int6-1F | GAAAGGCCATGCACCTAAC | 5-1R | CGGCTAGCTGTAAGATCTGA | <400>107 |
| <400>83 | IL-12TAQF | TAGCTCATCTTGGAGCGAAT | Int5R | CATGGAACTAAGCTGAGCCC | <400>108 |
| <400>84 | #6 | TTTGGAGGAAAAGTGGAAGA | IL-12 * R | CGCAGAATGTCAGGGAGAAG | <400>109 |
| <400>85 | T7 | AATACGACTCACTATAG | Int6R | TCCAGGTGCACTGAGAGT | <400>110 |
| | | | Int6-1R | TTCTAGCACAATTGCCTTGCCT | <400>111 |
| | | | IL-12B | AACATTCCATACATCCTGGC | <400>112 |
| | | | Ex8-1R | GCAGGAAGACACTGACTTTG | <400>113 |
| | | | Ex8-2R | GCCTTCCAGACACTTACGGT | <400>114 |
| | | | T3 | ATTAACCCTCACTAAAG | <400>115 |

LEGEND. Primers used for determining sequence from the PAC template, and amplification of genomic segments are listed in 5′->3′ orientation.

TABLE 13
Analyses of allelic transmission to affected offspring
TDT of polymorphisms in IL12B

| Polymorphism | Allele | Freq. | Trans | Not | P |
|---|---|---|---|---|---|
| 3′ UTR | 1 | 0.79 | 171 | 122 | 0.0025 |
| | 2 | 0.21 | 122 | 171 | — |
| intron 4 | 1 | 0.79 | 137 | 85 | 0.00029 |
| | 2 | 0.20 | 85 | 131 | — |
| | 3 | 0.01 | 0 | 6 | — |
| promoter | 1 | 0.56 | 176 | 191 | 0.77 |
| | 2 | 0.44 | 191 | 176 | — |

TDT at a centromeric locus, D5S2937

| Allele | Freq | Trans. | Not | P |
|---|---|---|---|---|
| 1 | 0.02 | 4 | 6 | — |
| 2 | 0.07 | 30 | 14 | 0.011 |
| 3 | 0.10 | 30 | 50 | — |
| 4 | 0.23 | 67 | 76 | — |
| 5 | 0.07 | 19 | 14 | — |
| 6 | 0.17 | 63 | 61 | — |
| 7 | 0.04 | 17 | 10 | — |
| 8 | 0.21 | 74 | 66 | — |
| 9 | 0.09 | 24 | 31 | — |

TDT allowing for multiple affected family members (Tₛₚ)

| Marker | DF | χ² | P |
|---|---|---|---|
| D5S2937 | 8 | 18.292 | 0.0191 |
| 3′ UTR | 1 | 12.694 | 0.0004 |
| Intron 4 | 2 | 10.549 | 0.0051 |
| promoter | 1 | 0.305 | 0.5809 |

TDT analyses (Spielman et al., 1993) of polymorphisms in and around IL12B were carried out on the data from the linkage study (FIG. 3). Markers are shown in order from centromere to telomere (Huang et al., in press). The D5S2937 marker is a simple sequence repeat which was generated from inspection of the draft sequence of a cosmid from 5q33-34. This marker was placed on the physical map in relation to IL12B. Note that no correction was made for testing multiple alleles at this locus. For clarity , only P<0.1 is shown. The data were used to calculate the Tₛₚ statistics shown, which corrects for multiple affected individuals per family (Martin et al., 1998). DF, degrees of freedom. No correction was made for testing multiple alleles at the D5S2937 locus.

TABLE 14
TDT of an independent cohort of simplex families

| Polymorphism | Allele | Freq | Trans | Not Trans. | P |
|---|---|---|---|---|---|
| IL12B promoter | 1 | 0.5 | 101 | 96 | — |
| | 2 | 0.5 | 96 | 101 | — |
| IL12B 3′ UTR | 1 | 0.78 | 101 | 55 | 0.00014 |
| | 2 | 0.22 | 55 | 101 | — |
| D5S2940 | 1 | 0.04 | 7 | 12 | — |
| | 2 | 0.5 | 93 | 97 | — |
| | 3 | 0.45 | 98 | 89 | — |
| | 4 | 0.01 | 5 | 5 | — |

235 simplex families with one affected child were genotyped for the IL12B promoter or 3′ UTR alleles, as well as for D5S2940. Note that although 235 families were tested, the high homozygosity rate for the 3′ UTR polymorphism meant that most parents were not informative for this marker. Allele frequencies were calculated based on parental genotypes. For clarity, only P<0.1 is shown.

### BIBLIOGRAPHY

Bennett S, Todd J. Ann Rev Genet 30: 343-70 (1996)

Boehnke M. et al., Am J Hum Genet 49: 1174-1188 (1991)

Degli-Esposti, M., et al., Hum. Immunol., 38:3-16 (1993)

Fu, J., et al., Diabetologia 41:228-232 (1998)

Gubler U. et al., Proc. Natl. Acad. Sci. U.S.A. 88: 4143-4147 (1991)

Hicks A. A. et al., Genomics 20: 285-288 (1994)

Huang, D., et al., Genes Immunity (in press)

Ioannou P. A., et al., Nat Genet 6: 84-89 (1994)

Johnson K. J., et al., Genomics., 14: 745-748 (1992)

Katz, J., et al., Science., 268:1185-1188 (1995)

Kruglyak, L. & Lander, E., Am. J Hum. Genet., 57:439-454 (1995)

Larsen, Z., et al., Autoimmunity 31:35-42 (1999)

LeCam, A & Legraverend, C., Eur. J. Biochem., 231:620-627 (1995)

Ma X, et al., Journal of Experimental Medicine 183: 147-157 (1996)

Martin, E., et al., Am. J. Hum. Genet 61:439-448 (1998)

Morahan, G., et al., Science 272:1811-1813 (1996)

Osoegawa K. et al., Genomics 52: 1-8 (1998)

Ramarao C S, Kincade-Denker J M, Perez D M, Gaivin R J, Riek R P, Graham R M. Journal of Biological Chemistry 1992; 267

Risch N Am J Hum Genet 40: 1-14 (1987)

Robinson, W., et al., Genetic Epidemiol 10:273-288 (1993)

Sambrook J, Fritsch, E F & Maniatis T 2nd ed. Molecular Cloning. (Cold Spring Harbour Laboratory Press: New York, ed. 2, 1989), pp. Pages.

Spielman R., et al., Am. J. Hum. Genet. 52:506-516 (1993)

Szelachowska, M., et al., Horm. Metab. Res., 29:168-171 (1997)

Tone Y., et al., Eur. J. Immunol. 26:1222-27 (1996)

Trembleau, S., et al., J. Exp. Med., 181:817-821 (1995)

Trembleau, S., et al., Eur. J. Immunol., 27:2330-2339 (1997)

Trinchieri G. Ann Rev Immunol 13:251-276 (1995)

Warrington J. A. & Bengtsson U., Genomics 24: 395-398 (1994)

1. A method for determining the presence of a disease condition or a predisposition for the development of a disease condition in a mammalian animal said method comprising screening for the presence of a form of IL-12 p40 genetic sequence or derivative thereof or its expression product wherein the presence of said form of IL12 p40 genetic sequence or derivative thereof or its expression product is indicative of the presence of the disease condition or the propensity to develop said disease condition.

2. A method for determining resistence to a disease condition in a mammalian animal said method comprising screening for the presence of a form of IL-12 p40 genetic sequence or derivative thereof or its expression product wherein the presence of said form of IL-12 p40 genetic sequence or derivative thereof or its expression product is indicative of resistance to developing said disease condition.

3. The method according to claim 1 or 2 wherein said disease condition is characterised, exacerbated or otherwise associated with Th1/Th2 dysregulation.

4. The method according to claim 3 wherein said form of IL-12 p40 genetic sequence comprises the Taq1⁺ or Taq1⁻ form of said sequence.

5. The method according to claim 4 wherein said form of IL-12 p40 genetic sequence comprises the nucleotide sequence substantially as set forth in<400>1 or <400>2.

6. The method according to claim 3 wherein said form of IL-12 p40 genetic sequence comprises a promoter region polymorphism.

7. The method according to claim 6 wherein said form of IL-12 p40 promoter region sequence comprises the nucleotide sequence substantially as set forth in any one or more of <400>3, <400>4, <400>5, <400>6, <400>7, <400>8, <400>132 or <400>133.

8. The method according to claim 3 wherein said form of IL-12 p40 genetic sequence comprises a polymorphism in exon 6.

9. The method according to claim 8 wherein said form of IL-12 p40 exon 6 sequence comprises the nucleotide sequence substantially as set forth in any one or more of <400>9 or <400>10.

10. The method according to claim 3 wherein said form of 1L-12 p40 genetic sequence comprises a polymorphism in exon 7.

11. The method according to claim 10 wherein said form of IL-12 p40 exon 7 sequence comprises the nucleotide sequence substantially as set forth in any one or more of <400>11 or <400>12.

12. The method according to claim 3 wherein said form of IL-12 p40 genetic sequence comprises a polymorphism in exon 8.

13. The method according to claim 12 wherein said form of IL-12 p40 exon 8 sequence comprises the nucleotide sequence substantially as set forth in any one or more of <400>13-<400>14.

14. The method according to claim 3 wherein said form of IL-12 p40 genetic sequence comprises a polymorphism in intron 1.

15. The method according to claim 14 wherein said form of IL-12 p40 intron 1 sequence comprises the nucleotide sequence substantially as set forth in any one or more of <400>41-<400>48.

16. The method according to claim 3 wherein said form of IL-12 p40 genetic sequence comprises a polymorphism in intron 2.

17. The method according to claim 16 wherein said form of IL-12 p40 intron 2 sequence comprises the nucleotide sequence substantially as set forth in any one or more of <400>49-<400>52.

18. The method according to claim 3 wherein said form of IL-12 p40 genetic sequence comprises a polymorphism in intron 4.

19. The method according to claim 18 wherein said form of 1L-12 p40 intron 4 sequence comprises the nucleotide sequence substantially as set forth in any one or more of <400>55-<400>58.

20. The method according to claim 3 wherein said form of IL-12 p40 genetic sequence comprises a polymorphism in intron 7.

21. The method according to claim 20 wherein said form of IL-12 p40 intron 7 sequence comprises the nucleotide sequence substantially as set forth in any one or more of <400>59 or <400>60.

22. The method according to any one of claims 3-21 wherein said disease condition is an autoimmune disease condition.

23. The method according to claim 1 wherein said disease condition is IDDM and said form of 1L-12 p40 genetic sequence comprises the nucleotide sequence substantially as set forth in<400>2.

24. The method according to claim 2 wherein said disease condition is IDDM and said form of IL-12 p40 genetic sequence comprises the nucleotide sequence substantially as set forth in <400>1.

25. A method of determining the presence of a disease condition characterised by Th1/Th2 dysregulation or a predisposition to the development of a disease condition characterised by Th1/Th2 dysregulation in a mammalian animal said method comprising screening for the presence of an allelic form of IL-12 p40 genetic sequence or derivative thereof or its expression product wherein said allelic form of IL-12 p40 genetic sequence or derivative thereof is linked to another gene.

26. The method according to claim 25 wherein said disease condition is an autoimmune disease condition.

27. The method according to claim 26 wherein said IL-12 p40 genetic sequence comprises the nucleotide sequence substantially as set forth in <400>2 and said disease condition is IDDM.

28. The method according to claim 27 wherein said other gene is a GABRA allele.

29. The method according to claim 28 wherein said GABRA allele is the GABRA1-A allele.

30. A method of determining resistance to a disease condition characterised by Th1/Th2 dysregulation in a mammalian animal said method comprising screening for the presence of an allelic form of IL-12 p40 genetic sequence or derivative thereof or its expression product wherein said allelic form of IL-12 p40 genetic sequence or derivative thereof is linked to another gene.

31. The method according to claim 30 wherein said disease condition is an autoimmune disease condition.

32. The method according to claim 31 wherein said IL-12 p40 genetic sequence comprises the nucleotide sequence substantially as set forth in <400>1 and said disease condition is IDDM.

33. The method according to claim 32 wherein said other gene is a GABRA allele.

34. The method according to claim 33 wherein said GABRA allele is the GABRA1-A allele.

35. A kit for determining the presence of a disease condition or a predisposition to the development of a disease condition in a mammalian animal said kit comprising a means of detecting the presence or absence of a form of IL12p40 genetic sequence or derivative thereof ot its expression product.

36. A kit for determining the presence of a disease condition or a predisposition to the development of a disease condition in a mammalian animal said kit comprising in compartmental form a first compartment adapted to contain an agent for detecting the Taq¹⁻ form of IL-12 p40 genetic sequence or derivative thereof or its expression product and a second compartment adapted to contain reagents useful for facilitating the detection by the agent in the first compartment.

37. A kit for determining resistance to a disease condition in a mammalian animal said kit comprising in compartmental form a first compartment adapted to contain an agent for detecting the Taq+form of IL-12 p40 genetic sequence or derivative thereof or its expression product and a second compartment adapted to contain reagents useful for facilitating the detection by the agent in the first compartment.

38. A method of treatment and/or prophylaxis of the disease condition characterised by Th1/Th2 dysregulation said method comprising administering to a mammal an effective amount of a form of IL-12 p40 genetic sequence or derivative, agonist or antagonist thereof or its expression product or derivative, antagonist or agonist thereof or a molecule which regulates the functioning of said IL-12 p40 genetic sequence wherein said IL-12 p40 or regulatory molecule thereof promotes resistance to said disease condition.

39. The method according to claim 37 wherein said disease condition is IDDM and said IL-12 p40 genetic sequence comprises the nucleotide sequence substantially as set forth in <400>1.


SEQUENCE LISTING (140 sequences)

SEQ ID NO: 1 - DNA, 419 bp, mammalian
cagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttcgatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatcca

SEQ ID NO: 2 - DNA, 419 bp, mammalian
cagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatcca

SEQ ID NO: 3 - DNA, 30 bp, mammalian
agttttttttttttaattttcaaggtgctt

SEQ ID NO: 4 - DNA, 30 bp, mammalian
agttttttttttttaaatttcaaggtgctt

SEQ ID NO: 5 - DNA, 30 bp, mammalian
aacatacctgcaatctgctttgtccactta

SEQ ID NO: 6 - DNA, 30 bp, mammalian
aacatacctgcaatctgatttgtccactta

SEQ ID NO: 7 - DNA, 29 bp, mammalian
ctaaaccctttgcccttcatctcatcctc

SEQ ID NO: 8 - DNA, 28 bp, mammalian
ctaaaccctttgccttcatctcatcctc

SEQ ID NO: 9 - DNA, 30 bp, mammalian
tcaaacctgacccacccaagaacttgcagc

SEQ ID NO: 10 - DNA, 30 bp, mammalian
tcaaacctgacccacccaacaacttgcagc

SEQ ID NO: 11 - DNA, 30 bp, mammalian
agatagagtcttcacggacaagacctcagc

SEQ ID NO: 12 - DNA, 30 bp, mammalian
agatagagtcttcaccgacaagacctcagc

SEQ ID NO: 13 - DNA, 24 bp, mammalian
ttgtatagttagatgctaaatgct

SEQ ID NO: 14 - DNA, 24 bp, mammalian
ttgtatagttcgatgctaaatgct

SEQ ID NO: 15 - DNA, 18 bp, mammalian
ggcttaaaggggccaagt

SEQ ID NO: 16 - DNA, 20 bp, mammalian
agggagcactatccctcagc

SEQ ID NO: 17 - DNA, 18 bp, mammalian
ggcttaaaggggccaagt

SEQ ID NO: 18 - DNA, 20 bp, mammalian
agggagcactatccctcagc

SEQ ID NO: 19 - DNA, 20 bp, mammalian
atgttatctcattgcctttc

SEQ ID NO: 20 - DNA, 20 bp, mammalian
aagtggttctgaaaccactg

SEQ ID NO: 21 - DNA, 20 bp, mammalian
atgttatctcattgcctttc

SEQ ID NO: 22 - DNA, 20 bp, mammalian
aagtggttctgaaaccactg

SEQ ID NO: 23 - DNA, 20 bp, mammalian
gggaagactaagctctactg

SEQ ID NO: 24 - DNA, 20 bp, mammalian
ggatttcgttccctctgttt

SEQ ID NO: 25 - DNA, 20 bp, mammalian
gggaagactaagctctactg

SEQ ID NO: 26 - DNA, 20 bp, mammalian
caacgaaccaagactgtcat

SEQ ID NO: 27 - DNA, 20 bp, mammalian
gggaagactaagctctactg

SEQ ID NO: 28 - DNA, 20 bp, mammalian
caacgaaccaagactgtcat

SEQ ID NO: 29 - DNA, 20 bp, mammalian
ttctaagccattcgctcctg

SEQ ID NO: 30 - DNA, 20 bp, mammalian
gttaattcattacactcacc

SEQ ID NO: 31 - DNA, 20 bp, mammalian
tacttctgctgacaccacta

SEQ ID NO: 32 - DNA, 22 bp, mammalian
gaactaggatcaaattgtatac

SEQ ID NO: 33 - DNA, 19 bp, mammalian
ggttacataatcatatgta

SEQ ID NO: 34 - DNA, 20 bp, mammalian
gttaggatttcaggtgtgag

SEQ ID NO: 35 - DNA, 20 bp, mammalian
tagctcatcttggagcgaat

SEQ ID NO: 36 - DNA, 20 bp, mammalian
aacattccatacatcctggc

SEQ ID NO: 37 - DNA, 19 bp, mammalian
gaaaggccatgcacctaac

SEQ ID NO: 38 - DNA, 18 bp, mammalian
tccaggtgcactgagagt

SEQ ID NO: 39 - DNA, 20 bp, mammalian
tttggaggaaaagtggaaga

SEQ ID NO: 40 - DNA, 20 bp, mammalian
aacattccatacatcctggc

SEQ ID NO: 41 - DNA, 22 bp, mammalian
tctttaataataactccctttt

SEQ ID NO: 42 - DNA, 22 bp, mammalian
tctttaatagtaactccctttt

SEQ ID NO: 43 - DNA, 20 bp, mammalian
gcccacccaagtgtcattgg

SEQ ID NO: 44 - DNA, 20 bp, mammalian
gcccacccaagcgtcattgg

SEQ ID NO: 45 - DNA, 18 bp, mammalian
tcaagcctgtctgtttaa

SEQ ID NO: 46 - DNA, 20 bp, mammalian
tcaagcctgtgtctgtttaa

SEQ ID NO: 47 - DNA, 17 bp, mammalian
ccgcctagacttagtag

SEQ ID NO: 48 - DNA, 17 bp, mammalian
ccgcctagagttagtag

SEQ ID NO: 49 - DNA, 32 bp, mammalian
taaaaataataataataataataataataatg

SEQ ID NO: 50 - DNA, 29 bp, mammalian
taaaaataataataataataataataatg

SEQ ID NO: 51 - DNA, 28 bp, mammalian
ctcctcagtctataagtaacaataacta

SEQ ID NO: 52 - DNA, 28 bp, mammalian
ctcctcggtctataagtaacgataacta

SEQ ID NO: 53 - DNA, 29 bp, mammalian
cgctcataagggttaaaaacaacaacaac

SEQ ID NO: 54 - DNA, 29 bp, mammalian
cgctcataagagttaaaaacaacaacaac

SEQ ID NO: 55 - DNA, 30 bp, mammalian
tctccaagtgcaaaaagacataatcagcag

SEQ ID NO: 56 - DNA, 30 bp, mammalian
tctccaagtgcataaagacataatcagcag

SEQ ID NO: 57 - DNA, 25 bp, mammalian
tatatatatataaaatgtgtataca

SEQ ID NO: 58 - DNA, 29 bp, mammalian
tatatatatatatataaaatgtgtataca

SEQ ID NO: 59 - DNA, 19 bp, mammalian
agagcatggaggacttgca

SEQ ID NO: 60 - DNA, 19 bp, mammalian
agagcatggcggacttgca

SEQ ID NO: 61 - DNA, 19 bp, mammalian
ggaaggcgccccagatgta

SEQ ID NO: 62 - DNA, 20 bp, mammalian
tcagacacattaaccttgca

SEQ ID NO: 63 - DNA, 22 bp, mammalian
caatagacaagtgatttcactg

SEQ ID NO: 64 - DNA, 22 bp, mammalian
atgcttaattataactatattc

SEQ ID NO: 65 - DNA, 20 bp, mammalian
ccataataggttcattgccc

SEQ ID NO: 66 - DNA, 18 bp, mammalian
ggcttaaaggggccaagt

SEQ ID NO: 67 - DNA, 18 bp, mammalian
cccaccaccatcacctct

SEQ ID NO: 68 - DNA, 20 bp, mammalian
atgttatctcattgcctttc

SEQ ID NO: 69 - DNA, 20 bp, mammalian
gaagaaaggggagaatcaag

SEQ ID NO: 70 - DNA, 20 bp, mammalian
ggaaaatgcaatgccatatc

SEQ ID NO: 71 - DNA, 20 bp, mammalian
atcctgaatttcctcaactg

SEQ ID NO: 72 - DNA, 20 bp, mammalian
agagactgtctgtatcccat

SEQ ID NO: 73 - DNA, 19 bp, mammalian
aggcctgagccaggggtat

SEQ ID NO: 74 - DNA, 20 bp, mammalian
ttctaagccattcgctcctg

SEQ ID NO: 75 - DNA, 20 bp, mammalian
attctggacgtttcacctgc

SEQ ID NO: 76 - DNA, 20 bp, mammalian
tacttctgctgacaccacta

SEQ ID NO: 77 - DNA, 20 bp, mammalian
ggatgaaggagacatacact

SEQ ID NO: 78 - DNA, 20 bp, mammalian
gccgttcacaagctcaagta

SEQ ID NO: 79 - DNA, 20 bp, mammalian
ggacttctttcttagaatat

SEQ ID NO: 80 - DNA, 20 bp, mammalian
atcaaacctgacccacccaa

SEQ ID NO: 81 - DNA, 20 bp, mammalian
atgtgatccttctttgactg

SEQ ID NO: 82 - DNA, 19 bp, mammalian
gaaaggccatgcacctaac

SEQ ID NO: 83 - DNA, 20 bp, mammalian
tagctcatcttggagcgaat

SEQ ID NO: 84 - DNA, 20 bp, mammalian
tttggaggaaaagtggaaga

SEQ ID NO: 85 - DNA, 17 bp, mammalian
aatacgactcactatag

SEQ ID NO: 86 - DNA, 20 bp, mammalian
gaattgcatgctgggttctg

SEQ ID NO: 87 - DNA, 20 bp, mammalian
ctgtggcttccagaggttac

SEQ ID NO: 88 - DNA, 20 bp, mammalian
taatgtggtcattggcaggt

SEQ ID NO: 89 - DNA, 20 bp, mammalian
cagatgagtccttgtgcccc

SEQ ID NO: 90 - DNA, 18 bp, mammalian
acccgggccagagcagcg

SEQ ID NO: 91 - DNA, 20 bp, mammalian
gcggaataaagatatctctc

SEQ ID NO: 92 - DNA, 19 bp, mammalian
gatgagatgaaggcaaagg

SEQ ID NO: 93 - DNA, 20 bp, mammalian
tcaccagggatgcttccagg

SEQ ID NO: 94 - DNA, 18 bp, mammalian
ggttacccacattccatc

SEQ ID NO: 95 - DNA, 20 bp, mammalian
cttatgccatggatcatgtc

SEQ ID NO: 96 - DNA, 20 bp, mammalian
aagtggttctgaaaccactg

SEQ ID NO: 97 - DNA, 19 bp, mammalian
ccagagtgtctgattcagc

SEQ ID NO: 98 - DNA, 20 bp, mammalian
aaggcaagccatctgataca

SEQ ID NO: 99 - DNA, 20 bp, mammalian
aggccaacgatctaagcatg

SEQ ID NO: 100 - DNA, 18 bp, mammalian
ggagtggcagaggcctgg

SEQ ID NO: 101 - DNA, 19 bp, mammalian
caccatttctccaggggca

SEQ ID NO: 102 - DNA, 20 bp, mammalian
caacgaaccaagactgtcat

SEQ ID NO: 103 - DNA, 20 bp, mammalian
gttaattcattacactcacc

SEQ ID NO: 104 - DNA, 20 bp, mammalian
ataggtcactgagaggttgc

SEQ ID NO: 105 - DNA, 20 bp, mammalian
agcttgttgtatccttccag

SEQ ID NO: 106 - DNA, 20 bp, mammalian
tcatactccttgttgtcccc

SEQ ID NO: 107 - DNA, 20 bp, mammalian
cggctagctgtaagatctga

SEQ ID NO: 108 - DNA, 19 bp, mammalian
catggaactaagctgagcc

SEQ ID NO: 109 - DNA, 20 bp, mammalian
cgcagaatgtcagggagaag

SEQ ID NO: 110 - DNA, 18 bp, mammalian
tccaggtgcactgagagt

SEQ ID NO: 111 - DNA, 22 bp, mammalian
ttctagcacaattgccttgcct

SEQ ID NO: 112 - DNA, 20 bp, mammalian
aacattccatacatcctggc

SEQ ID NO: 113 - DNA, 20 bp, mammalian
gcaggaagacactgactttg

SEQ ID NO: 114 - DNA, 20 bp, mammalian
gccttccagacacttacggt

SEQ ID NO: 115 - DNA, 17 bp, mammalian
attaaccctcactaaag

SEQ ID NO: 116 - DNA, 24 bp, mammalian
gcccagagcaaggtaagcacttcc

SEQ ID NO: 117 - DNA, 24 bp, mammalian
cccttcttatagatgtgtcaccag

SEQ ID NO: 118 - DNA, 24 bp, mammalian
tgaagaaagatggtaaccagcctc

SEQ ID NO: 119 - DNA, 24 bp, mammalian
tgtgcattccagtttatgtcgtag

SEQ ID NO: 120 - DNA, 24 bp, mammalian
aggaccagaaaggtaattctatac

SEQ ID NO: 121 - DNA, 24 bp, mammalian
tttcaaatccagaacccaaaaata

SEQ ID NO: 122 - DNA, 24 bp, mammalian
aagcagcagagggtgagtgaaact

SEQ ID NO: 123 - DNA, 24 bp, mammalian
ctttgacttcagctcttctgaccc

SEQ ID NO: 124 - DNA, 24 bp, mammalian
tcagggacatcagtgagttttgga

SEQ ID NO: 125 - DNA, 24 bp, mammalian
cctcttccacagtcaaacctgacc

SEQ ID NO: 126 - DNA, 24 bp, mammalian
aagagagaaaaggtaagaagtgat

SEQ ID NO: 127 - DNA, 24 bp, mammalian
tctcttttgcagaaagatagagtc

SEQ ID NO: 128 - DNA, 24 bp, mammalian
ccctgcagttaggtgagcaggccc

SEQ ID NO: 129 - DNA, 24 bp, mammalian
attctcttccaggttctgatccag

SEQ ID NO: 130 - DNA, 18340 bp, mammalian
caatagacaagtgatttcactgcgggaagacaattcagagccctgttccaggctcctcacattgattctctctgtcttcttccactcctctttgtcatctttgatgtccccttgtgagctacgaaaagactttctgggacacgacaggataaaaaaataaataagtgcaagcagccattcattaaacgtttagccaggatgctgctttaactgcatcccatcatatctcattaatcttcacaccagtcctgagatcaggtactattattaacccgattttacagatgtgaggaactgaggcttaacgaaggtaagtaacttgcaggtgcgggtatccagctctctaactccagagcccatgctcttaaaaccctattacttgtccctggtggaggtgaacactgggggccctttcatataggactagccctcgggctgcaatctgagcggaaaagggaggatgagggcatacttcgaagcttcttttgcataactggcgctggatttttactgagactttacgttacagttttttttttttaattttcaaggtgcttttacgaacacatgaataaaatatttgtgtcattttgaaccttacttgtcttattttatgcatgtatttatttatgggggggcacaaggactcatctgtggtggtgcagccactgtaaataaattagtgaaactacttcacgtcaatttctgttcagtacactttagtgatggatcggaggaaattaatacatgtttacaaaaagcccctcccccagttgttacatatgcctcagagataccagttgtgaaaagtgcaggtgcacttacacacatacgcacacacaccccacaaatggtatcatacgaaaaaacatacctgcaatctgctttgtccacttaattgtatatcttggatacagaacttgtttcactggaaggctaaaaggcaaagtctggggaggcctagaggacacaggggatgggaggaggcgctctgagctggatgtaaggtctccacccacggccagagcacaaggtcggataaccagtgggcctgccggcttggctgcctgggccctcccctgccgagacaaacggctggagggaggaagtgtgcggctgggaagctccgctgctctggcccgggtttcccatttcccccttcccgcgctgagacggcgaggaaagttagcccggaaatctgcgcccgcctaaaacccggcctggtcccagccaccgccccaggaacttcccccaccgcaggggcggaggtcgagagcagggatggagaagtggacctgcgcgggtggactccggggcgcgggtggactccggggcgcggggggactccgaggagcgggtggactgtggggcgcgggtaccgtctcgcagcgacctctgtcggcggctctggggatggcccgcatctgtctgcgtgtacctggtatacgtgcaggtacatgttcctgttcacgtgcagactgggcgggggatgggggggtccacaccggtgtacacctttgcatacctcttagcaacttgaaattccaccacgagagatatctttattccgctattcctgtgcatctgcacggagcccctagggccatagatttgtgtgcaaatgaaatgaggatgtagtctgggtgcccaagggggggtgccttgagtgtggttgtctgtatgcctccctgagggtatttcactttctgctcccatccgcccctatgagcgagtacctatgagcacaggatgtgcacatatttgagtcttattagtggtacacgcagttttatcatctccccaggtctgtgtctgtatgaaatgtgcatgggtgtgtgtgtgcacgcgtgtgttcccactcggggaatgtggggagaggtgcatggagccaagatgggtggtaaatagtatgtttctgaaattaaaggactaatgtggaggaaggcgccccagatgtactaaaccctttgcccttcatctcatcctctctgacttgggaagaaccaggattttgtttttaagcccttgggcatacagttgttccatcccgacatgaactcagcctcccgtctgaccgccccttggccttccttcttcctcgatctgtggaacccagggaatctgcctagtgctgtctccaagcaccttggccatgatgtaaacccagagaaattagcatctccatctccttccttattccccacccaaaagtcatttcctcttagttcattacctgggattttgatgtctatgttccctcctcgttattgatacacacacagagagagacaaacaaaaaaggaacttcttgaaattcccccagaaggttttgagagttgttttcaatgttgcaacaagtcagtttctagtttaagtttccatcagaaaggagtagagtatataagttccagtaccagcaacagcagcagaagaaacaacatctgtttcagggccattggactctccgtcctgcccagagcaaggtaagcacttcccaagcccctacctccctcccctccctgtgggcctgcagctgtccaggtgtagaaaccgttagtgtgctaccccagcagctggcaggagggagttggtggattcctggaagcatccctggtgagtcatctgctggaacattagtgaaaacttagtactctagggaccgatgtacagtgtccattttaaaagccacctaataataactgtatagcaagatctgtgtgtatgcatagtttgtggaaatgtttgttttatcttattttgaagtggtgtgtattgatgtataaaagtatattcctaaatgttaatgcccatcagttaaaggattgtatagagctaaagtgagtggtgcctgccttactattgaaatttttaaaaagcctttcgtgcattccttaaagtaattggattcataattataataatgttacaaatcagacttgctcccatatttgtgatggtcttggtcgtcagttgtgatatcaaccaaaatgacagctgggatccccattcttgtggattaactaactttggccccagttaaaaaatgaaaagctattattgcttcctaaagagtttttaaatctgtgagaagggggaaaaaaaggtttttttacttgcccaggtaaaattgtgtgcaacaacaatgtcattttaacaaagggattactaaaccccaggtgatgacccactttttcaaacaaggaattgcaagatactagatggaatgtgggtaacctcttagagtttagtttactggaatctgaaattgtatgatttccgtacattccttcatgctactaataagtcatggaatccctctgtgctggactctgggggtaaagtgcaaaacaagatagacatgatccatggcataagagagttcactcagtgtggcagagaagacaaacagtaaagaagagactgtattgttgccattgtagtaaatgctgtggaaggaggggagcaaatagtgggtcctgacttcatctaggcaccatgacacttcacgtgaattatgtcactaactccttaccacagtagcatgcccattttatagatcaggagtgtggggcttaaaggggccaagtgactcacccagagtcacacagttctagaagtctgcctggccctcaaactagggatctttgcatgtgccacccatgccatctgtgactatatctttttattctttaatagtaactcccttttctaattaaaggtaacaaacaaaacttaaaaaagagatgcccacccaagcgtcattggcatgctgatgttggcaccagtgttgggaagcccttagcatactccaggaagtaggagtgtgtaacgtggggtccctttgtccttcatgcaagggtttcaagagtttagaaaacctatgaaattgcacacacaaaaatgtgttttaatcatcaagactctcagacttaccatgctgagaaatgtgggctgagggatagtgctccctagtatcagctgatgggccagagagccaaaggaggagacccaccaccatcacctctcctggacagtgctctgtggtttcaaatgtaggtgatactaaaatgggagttgtttcttagaaccatggccggggttcccttgaccctgaagtgcagttcacctgagattgtcaaatgcatctgaggcatgcagaggaagtgctgggcacacagctagtgggaatacctcagcgtaagtggccaggagatgccaggaatctccactatttcccttccagtgtgccagcctctgggttttacaggcgcatgtaattgcagtacctctgtgcacatttccctactgcctagaatgactttcttgactatccatgatatataaaacacagataccaaattgttcccttacctcttcctctaggttcaagttaatatgttattggttgccttctataatatgttatctcattgcctttcccaacaagtctttgagataagtattaggtccattttatagacaaagagactgaggctcagcgagtaacttggccaacaagttgctcccactgctcaacagcaaatgagcggtgggaccaaaattcaagcctgtgtctgtttaacttcaagcctgtgaatgtactaaccggtgccctgtgccagctagtactttgctacagtcataacctagactgaagtgatagccatgcccctaaaactccatgctgtggtgacagcactgagcagtgtccaagaaggcttgacttctaggcctgtctctgccactcacaaactttataagggaataaagtacatagcaaggtccgcctagagttagtagcagttctgacaaagctgtaatttgtcaatattccgtcacccaacccaggaatgctcatttttaaggtatttgactgaaacagttgagcattgcccttcatatagtttaaaacagtggtttcagaaccactttcctccagaccatgggtgctctgcaaggtgaatggagttgtttcagaatgtttcaataatcatccctacctcattcgtaagtggcatgtaatttttgcaatcggaagattttcataaaccctggatactaacctagactggtttctatatcagatggtggcttatttaacataaaattatgcattttactatttcatggtggatatatcaatatgttgtggtcttttcccaatgaacactttgattttcaggggttctggaccctgaacatgggttaaaccagtggttctcaaggtgtggtcttagcgccagcagcatctgcttcccctggaaactttctagaaatgcatattctcaggccctcatgcctgctgaatcagacactctgggggtgggactcagccgtctgttgtagcagtgcttccaggttatcctgacagtcactcaaattttagaaccactaggttctctatatgggagagagtagtctttgaacttggaaaacaagagaagctaaacccctacagcaagggctggtgaccaggtcgttgccagaacctgaaagttcgcctctgtattaccgttcctgtccctaacccaagtccttcagttctgggtgctccagcacacactgctttgtgctgcagtgatacaaatgtatggctcatctccccagctggcggggaggcatttaacacactgacttaataaatatttattgagtaaaagtatttgctcctaggaagcgggatccaggtaagccctttttttctctctcaactgcttctagcccagtgctctttatgtagtaagcactaaataaacaactgctagatgttgatccagaaagtcacattccttctctaagctttaagtttctcatcttaaaaataagaggattgtatcagatggcttgccttaggtctctttcagctccagagccccaaataccctatggttctctatttagagatgttcttccccacagactgccatagaactcctgtaatttacttagtatttgcttgacagtatggagaagaaaggggagaatcaagattttatttaaaaaaaaagtagctagaatgtgtatatggttcacaaaggtaacaagaattattgacattctttcttctcttttttcttcctcttccttctcttttcctccttctcttccccctgcttctctcccttcttatagatgtgtcaccagcagttggtcatctcttggttttccctggtttttctggcatctcccctcgtggccatatgggaactgaagaaagatggtaaccagcctctcattattctctgtggaggccccacttctaagccaggactcttgggcagccactggtgggaaatcaaactgaaatgggcaaccatgcactgggtcctctagagaaagccatcactctgggaaaatgcaatgccatatctctcttttctactttgatggtatctatattgtttggttttcacattggatgacattggtacactatggtggggaaagacatatgatatatgatatggtggggaaagacatatgacatatgatattttccaatattactaaaaactgtttcacacaattaaaattccaaagtagaggatttgcaaagtataacaactgtgttcgtttctcattccaccacatgatactgccccctcagttggcactgtgatgacttacctctgaccaagcactttggaggaagcataggattcagactcacattgacttgggttcaagtcctaggtctgtcaatgactggttatgtgactttaagctgggtcacctctaatcctgaatttcctcaactgtaaactggatgttacaaagtggatgcctaccacgtgggttatttagtgggttaatgaatgcagaatacaactcgacagatagtaaagtgaaagtaaatgtcagctagtattactattttggttgtttaaaatatctttcatgattcaagagatactttttattatcccaatgatcagtaaaaattattagtagactaatagaatagttaatggtaaaataaggagttctgcccatccttctagtatctcacactcagtaaatgtgcattctgaccgttggctgtacctgaaagaccctcagatttttatcactgaagccaacatcataatgttggcgattactatctttaattgtataataataatagttaatgtttattgagtgcactgtctcacttaattctcacaagagccatatgaagtagagactgtctgtatcccattttacagatgttggaaactgaggccagagagattaagtaacttgcccaatgtcacatacctggtaagggtggaacagggacttgatcccaattctgtcttgcttcaaagctggtgcacttaaatttgtgaaaacgtttttacaaggacatgaagtaattttttcccaggtctttggagagctgaataagaggaaatggacataaattaaggatgaaaatatttcagctgatgatcagaaataatcttttgatattctagaaagtaccatcttgaaatgggtacctgaaagaaatctgggaccactcctctcttctcaagaattttaggaagacagaatccagccaccccttctccatgagaatttgagagatttagacactctcttaagtaaaggcaaaggcctgagccaggggtatatggcagatcccttccaaccctgggattgttagcgagctcaggaaccttggtcctggcatatttgaccccttagtgacttctgatttggtaaaccacagaaattccagaaaatcagtgtgagaaactctctgaggtgtgacttaggagggcagacgatgcagtgaggctaagtgccaggttcttgatgctcctcttcagctttcctcctgcagctgttttccctgctgttgagcaaacatcttctagggcttccgagcctcagttgggacaggaaagtaaccatgctcttcaggtgtcagggggacaaaaaaaaaaccaagaaaaagccaaaagtgccacatggttttacatcagcacagctaatcatttccccagagttggaccccaaatgcttttgacctcttatttttgttatccattcagtccttataatccaattgatgtaaagtgaaaactttatattctacaatgctttacatccagaggccaataacgagaaccaccatttataaagcatgtaagggcactgtgtatgagcttatataatccacatatccaccttctaaagcaagggctaatatttttctcattttaaagatgatgacactgaggcttacagtagttgaatgtcttgccaaaggccacaagactggagcaagggctagagctgcttctaaatccaggcctctgccactccaaaatgcaggctctcaaccactgtgactcataaacttgagcaggcatcagcaccatctggagagcttaagaaccatataactaaatccatcccaaggtttctgattcagcagctgagaatttgcatttctgatctattccaaggtgatgctgctgatggtgtttcatcgatcatgctttgggaactactacattaaacaattctattcaattaataatttatgcatggattaaaaaaatgaatgaagctttgctatgacacactctgaaatactatactaagccattcctcaaaggccagtttagacactagcattaggcatcccttgcaaagcccaagagacaaaaggtctgagctatagcccttgtacttctgacttgctgtgaccatgcttagatcgttggcctcagtacgcttcttcattaaatgggaagactaagctctactggactgcttcataagagtgtaagatagctaaaaataataataataataataataatgcagagagaatgaaaatctccactggtgatttaaaacagagggaacgaaatccttaaatatccatggaaaattgttaagagagtttctctgtacagttggctgactcctcggtctataagtaacgataactaacaccgaatttactgtgtggcagacactgtgctaagtactttacgtgctttttttttttttcatttaatcctcagtcaaatgtaaggcagatactgttattattatcattttacagatgaggaaactgaggctcatgataatgaaataccttgttccaaatcccccagctggttagtggagacaggatgacagtcttggttcgttgttctcgacaccctgagcttttaaccactatgttactctgctgaatattgtgccctgccgtattctctatgaaactgaaattgtgctggaagtttctctcccccagacctttggcaaagagtcttgtgctgtttgcagtttttggtatattaaggtgtttccaatctgctaaataatcaaaggttactattaaaggcagccttccagtcaatgagtcgatggcagctataaaactctttgtttctcttttccatgaccttgagcccaagcagggtctcatgccttgagatcatctcagcaagcatttgccaaatacttgttgtaaacaaggttgtgtttaggcaatggggatgcccgaagggttaataaaacacagtcccagagttcctggagcttacagcctggttctccactttatgtgcattccagtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaaggtaattctatacccttggatagtatcaattttctctttcgctcataagagttaaaaacaacaacaacaacaaattgaaaagccaagtcatggtgagtgtaatgaattaacatcaagtctcttattgatgttaattgatgttaacctccattttcctttgctttcctggaccctttgggttatcaaccatcaaaatctcatattaagggagtttcatgatcagtctgaatgcttagcctcatgttttctttaaataatggtgatattatttaatggctaatggaaattaaccgatagtgtatcactctgcactggggtgatagccttcaaaaaatgaatgcctctgccaggcatgttaggtgtgtagtgtactctgcagaatcaacaccccactgggatactcccaatccttatggagctacccaagaggcaacgcatggaagaacttcaccctgtaccatctggtgatctgtgattcatcacaatcaaaacctttctgcaaaaaactcctaaatattgaatttttgtttttttcaaatccagaacccaaaaataagacctttctaagatgcgaggccaagaattattctggacgtttcacctgctggtggctgacgacaatcagtactgatttgacattcagtgtcaaaagcagcagagggtgagtgaaactgctctggtttctcagcatttttctagaactatttcattaagaaattaagggcaacctctcagtgacctatcagttaatgataatgggaaaagcaaagtcaaacccgtgttttttcaaccgcccttccttgtctacattgaagaaagaacatggagattttagccgattgcttgaataaatgtatgtgttggggcaggatattattgggaactgagaatagtctctgctgtgtttgaacccactcatccaaattgcctggccatgcttcctgaagcctcatagcaccaaagaaagggataaaaggagaattcaaagctacaaatgacttgctgaaattgcaccttgagtcaaaaataaaaacaagagctccagggcgtagatcttaggggccctgaagcagactccaaaactcgatgaggcctcccgaaattttcccagggccacctcaactccttttacttctgctgacaccactaatctgaagttcgctgttggtccaatgcacctggactttccgtaagaaagcaacttccataaatacaagacctatgtgttaacccccatgtggcttactttaatcatcaccgaagcaaaccccaggtgatcatcctgactttaccattatttcactgagtaaattaagcattggggtctcactttttcatctttaaaaggaaaatgcttactaaagaaatgtttctccaagtgcataaagacataatcagcagaggaatggttaaataaaacatggtacactatactcttgcttaatgtgcagtcattgaagtggataacccaacccatatgttttgtcatggagagctccccataatatgttcagaggggaaaaggatggttacataatcatatgtatacaatttgatcctagttcataaaaataaaatctatatgtataagtaaaatatatatagtggatatatataatgtagagatgtatataacatggattatatatataatgtgtgtatacatatgtgtgtgtgtgtgtgtgtatatatatatatatatatataaaatgtgtatacaattatcttgaatattcattgaaaaagttctggccaggcacagtggctcacacctgaaatcctaactctttgggaggctgagacagaatgattgcttgaggccaggagttcaagaccagcctaggcaacacagtgagaccccatctcagaaaatattaaaaataaaaaaattaggtgggtgtggtggcacacacctgtagtcccagctacttgggaggcagagggaggggatcacttgagcctaggagtttgaggctacagtggggtctgattccaccacttcactccagcctgggtggcagagcaaaaccctgtctcttaaaaaaaaaaagagagagagagagagaaagaaaaagaaaaaggaaggtctggaaggatacaacaagctattattagtacttaaacctgtggagagcagttaaggatgaaggagacatacacttctttcctttatatggatctttatcatctttacttttataattagtgtgtactgatttgtgtattgattttataattaaaatgggaaaaaatgaatttaagtttttaacaagggggtttaataatcagagattctagatctaaaacaaacaaaaacttccatattcatttagtccagagacatgtaagtgctcttgaatttaagctttttctcctggggagggcagtttcttaccctctgggtagaaatcagcccagttggagaaactgtgtcctcagacaacagttgaggccttacctgccttactggctacaatcactaggaactctctccccaatgtgtaacacaggctaatttctgtctttgacttcagctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcagtgagttttggatgattatatgtgctccataaggaaagatactatttgtcacgtgttcacaatgccccatgcactgtggggtaggtggttgacaagcatcatctcttttattctgcatccaaaaacaaaatacgatgtagatactgttatctgcattttaaggaagaggaaattgagtcttagaaaagttaagcaacttgccccagatctcagatcttacagctagccgttcaaatccagatccactccactacagctgctctttactgcactttgattcagctgccagatagtttccatgatgaatcccagagcctaatcaagcataatattcatattcagaaccagggcttccttactaatggcaattattcccaaccaatccttccttagcatttgaaaagggacttctttcttagaatataaacccttccaaaatggacatctttttttttaattggcagatagggatttcaccataagtcatttcctttactatttattcattgaccaggcagcatgataaagtgtaatagaaccagagaacttgcttcaaaacttatggagggtttgtacttggtgggtggggtctagttcacatagggtggccaaggaaggcctctctgaggaggtgacatttagctgacaccaaaaggaaagatgtcagttgtgttaagagcagagggaagcatatgtgcgaagcacctgctaggagccgtgatctttgtgtggagcagtgccaggcctacagagcccaaccacacaccctagcatgtctctgcctcctcttatctagaagacctaattgaggaaggagtctttgtgaaactcactgctgtatccttcatgcacagtccagtggctggaacataatgggcgctcagtattcatggaataaacaagcaaattgagcatagagacaattgactgtaactgctccaagacatgtccgcaccaaaagctatgaaaagacaaaagaaagggcagtaaatagaaaatctatcatctcatccccagggagaggctcagcttagttccatgttcagtgcaaagtgagggattagcacagacagggtggtccttcaatgcatggcccataaccattaaagcagaggtcttctcactgtgcggtcccatctgattgttcagtgatgaggattctgagcatctctcagatcctgcaatacatgtggatctgagatgtggccattgataatgactgccttcccgaggcaccagcgtgagcacctgcggcagaggtgcctcacatttgccagccaggtgctcacagaagttaagtaactatccagtggactcacagctgatcaaaggtgcaagtgagatcataagccaaaaccactgaactccaaagccttattaggaaaataaagcatgtttatcctcttccacagtcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaaggtaagaagtgattcaggtgcagtatattccttggtcagttttacggaggcccaccataaagtgagaagatgaatgatgataataacaatgacatccatgtatcacttaacaacagggatacattctgagaaattcatctttaggcagctatatcattgtgcaaacatatatggtgtacccacacaaacctagatggtatagcctactacgcttctaggctttatggtatagcctattgctcctaggctgcaaacctgtacagcatgttcctgtactgaatactgtaggcaattacaacacaatggtttgtatatctaaacagaaaagatatagcaaaaatacaatattataacaatataggaccactggtcatatatgtgatccttctttgactgaaatgttattatgtgatgcataattacttttcttagcacttttctatgtgtctagagctgtgccaagggttttccatgtttatttcacttaatctacaaaaattaacgcaacaaaggtagctgatgttattcttgtttttttacccccttttttgtggaaaagaggctttccttttttccagaaactgtggcaaggtaaagtaaagctgtagctgatgcaggaattttgtgtaggtgttagcagcactgccctcactacgtgctcattggacagtagcccaaccccaagaaaaggatggttggtagccagtagtattatcatcatttcacaagtgattgaagactcagagaggttaagtgactttaccaaggtcacccagctaggaaatgacataaccaagacataaactcaatctgccagacagaaaggccatgcacctaaccactccactacctctgatgttggtcattgatcttggcactcagaattagtcctgatagaggagacctgggctccagaagcctaaaattgttgtttcaactgagtgcatgtaatgaatgatagaacaggcaagagatatcgcccccaaaatggatagctcctggctgttccagatattataaaattattttactaaacagaatgtctacacttatagaggctaagatattggcttcccagcttcctcgccttacagcagaattcctttgcctgttgcaaggttccagaggcccttttgtaccgccccagactcctttcaccccacttttaaaatcactggacaaagccctaattcagcatagcatttagcatgtggtagaaattcagtgagctagttactctctgggaaaataattaggtagggaggctatcctggaatagatatttacctaaatattattttacatcttggcaagtactttccctatttaagatctgtatgactaataggtgatattgagtgcttcctatgtgctaaagacttgctaagagtttgacgtgatttttaccttgaactataattctatgaagtaggcattattgttatccctatttataagtgaggaaacagacacagagaacctaagacattttcctgaagttacacagctattaagtagcagtgccagaatttgaaggcaagttttctgatgaaatgatcaggatatggtatttctcaatatctcagggatggctagagcaaatctgtctctctctcaccatcagctcaggactgggtgagtggccatggggtcttgaggcaaggcaattgtgctagaaagatgaaagctgggccaaacgatttctccctcaagggcttacaaagtacaaaagctgcacctacatgtggagtgtctgccagtaggtggtgcaagttctatgcacacccctgtgaattgcaagcacagtgccctaagaccaagatgggcttgttttgggagagtatgcattgcagaaacaggctcagcttaccctgtgactatgttgccaaggggtcttcacagctttccttctcttttgcagaaagatagagtcttcacggacaagacctcagccacggtcatctgccgcaaaaatgccagcattagcgtgcgggcccaggaccgctactatagctcatcttggagcgaatgggcatctgtgccctgcagttaggtgagcaggccctcaaaggccagcccaggcctgcactctcagtgcacctggatgcagggatatgattgggggctgtgttggagaggaaagggggatggagtggccagcacccagttgccagaatcagaaacatacatttattcactaacagatatttatttggtgcctttgttatgtaggacactgtgctggccacagggatattgcaggaaagaaaacagaccggggttctggcctcctaaagagaaaggcaaagaaaagagagaggtagccaggaggcagagcatggcggacttgcaagcttgcaggactcagaatcttgttctgggggccccgggccctgaaacccactgaagggttttcagcaaggaagtaacacaatcagatattattttaagaaaaccctcaagaaagcctctggcaagcatggtgccagccaaattccaggccacataaggaaggcctgggccttctggcatgaaatccctgaaacccagttgcccaggatcatatgttgtgagaaataagaagagacattgctgttacaatgtcaccccacatcaacttttggcattctcttccaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccatgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatcatattttgggcattctcagctgatttgagtactctgcttgcaagtctaccaggggtgcattttttcccctatcatttaagcattgccctttcttgaagtgatcccattccaaattttgcagagttgctctttccccttatagtatttccaaagtcagtgtcttcctgagctcaggggatgtccctgtaatctgacaaggaaggatcc

SEQ ID NO: 131 - DNA, 1263 bp, mammalian
atttgtattgtttgaagtttttacaatagcatgtaattttctaagattttaatttttataagtacacatggcttctcctttatttgaaatgtgtactagatgatcaaagcatatgcatgcatgtattgctttcttctaggagaaaataagtttttgtggccaaaaaaattctttaagttatttttggtttttagggggttgcctccatgtcacagcttaatcatcagacacattaaccttgcagctcagcacgccctctgtttgtcagcagaccttcctcgcccatagggtaagcaatagaaagcttataggtatcagtttattttgcctgggatcagggtctggattgggaagtgggacatgttgataaacctcttctccaaaattaggtcaatgggcatttggctcatattaccagaatgctggctggccatgtacagcctgtctccgagagaggctctaatgtggcccccacattagaacaacctgccaatgaccacattagaacctccattgttaaaatgcaggttcctgagccccatcccagatctgaatcacaatctccaagcatcagccccaagaacctgaattttgttgttacatgcagataaagtacgagaaccacttcctccatgggtgaactgaacttaccaaaatagtcagtcccgaggggcagagatggcgtaggtgccagttcttctttctcatcctagatgctcagagtcaaattcttggctcagcaatagacaagtgatttcactgcgggaagacaattcagagccctgttccaggctcctcacattggatctctctgtcttcttccactcctctttgtcatctttgatgtccccttgtgagctacgaaaagactttctgggacacgacaggataaaaaaataaataagtgcaagctgccattcattaaacgtttagccaggatgctgctttaactgcatcccatcatatctcattaatcttcacaccagtcctgagatcaggtactattattaacccgattttacagatgtgaggaactgaggcttaacgaaggtaagtaacttgcaggtgcgggtatccagctctctaactccagagcccatgctcttaaaaccctattacttgtccctggtgggaggtgaacactgggggccctttcatataggactagccctcgggctgcaatctgagcggaaaagggaggatgaggggcatacttcgaagcttcttttgcataactggcgctgggatttttactgagacttt

SEQ ID NO: 132 - DNA, 49 bp, mammalian
tgtacagcctgtctccgagagaggctctaatgtggcccccacattagaa

SEQ ID NO: 133 - DNA, 45 bp, mammalian
tgtacagcctgtctccgagagagggctgtggcccccacattagaa

SEQ ID NO: 134 - DNA, 20 bp, mammalian
tagctcatcttggagcgaat

SEQ ID NO: 135 - DNA, 20 bp, mammalian
aacattccatacatcctggc

SEQ ID NO: 136 - DNA, 20 bp, mammalian
gccaggatgtatggaatgtt

SEQ ID NO: 137 - DNA, 20 bp, mammalian
gggtaagcgattcaaacatt

SEQ ID NO: 138 - DNA, 21 bp, mammalian
ggtattgcattgtaggcacat

SEQ ID NO: 139 - DNA, 19 bp, mammalian
gggcaacaagagtgaaact

SEQ ID NO: 140 - DNA, 20 bp, mammalian
tcaaaagaggtccgtctaaa